(19) United States
(12) Patent Application Publication
(10) Pub. No.: US 2014/0045908 A1
(43) Pub. Date: Feb. 13, 2014

(54) HSP90 INHIBITORY COMPOUNDS IN TREATING JAK/STAT SIGNALING-MEDIATED CANCERS

(71) Applicants: Ronald K. Blackman, Brookline, MA (US); Kevin P. Foley, Landsdale, PA (US); David Proia, Newton, MA (US)

(72) Inventors: Ronald K. Blackman, Brookline, MA (US); Kevin P. Foley, Landsdale, PA (US); David Proia, Newton, MA (US)

(73) Assignee: SYNTA PHARMACEUTICALS CORP., Lexington, MA (US)

(21) Appl. No.: 14/001,427

(22) PCT Filed: Feb. 24, 2012
(86) PCT No.: PCT/US12/26423

§ 371 Date: Nov. 4, 2013

Related U.S. Application Data
(60) Provisional application No. 61/446,682, filed on Feb. 25, 2011.

(51) Int. Cl. A61K 31/4196

(52) U.S. Cl. CPC A61K 31/4196
USPC 514/384; 435/375

(57) ABSTRACT

Methods for treating a subject with cancer mediated through dysregulated, aberrant, or defective JAK/STAT signaling, are provided, comprising determining the level of the JAK/STAT signaling in a sample derived from a subject in need of treatment, wherein the presence of dysregulated, aberrant, or defective JAK/STAT signaling is indicated, administering to the subject an effective amount of a triazolone compound as described herein.

### CROSS-REFERENCE TO RELATED PATENT APPLICATIONS

This application claims the benefit of priority to U.S. Provisional Patent Application No. 61/446,682, filed on Feb. 25, 2011. The content of the above referenced application is incorporated herein by reference in its entirety.

### BACKGROUND OF THE INVENTION

Although tremendous advances have been made in elucidating the genomic abnormalities that cause malignant cancer cells, currently available chemotherapy remains unsatisfactory, and the prognosis for the majority of patients diagnosed with cancer remains dismal. Most chemotherapeutic agents act on a specific molecular target thought to be involved in the development of the malignant phenotype. However, a complex network of signaling pathways regulate cell proliferation and the majority of malignant cancers are facilitated by multiple genetic abnormalities in these pathways. Therefore, it is less likely that a therapeutic agent that acts on one molecular target will be fully effective in curing a patient who has cancer.

Heat shock proteins (HSPs) are a class of chaperone proteins that are up-regulated in response to elevated temperature and other environmental stresses, such as ultraviolet light, nutrient deprivation, and oxygen deprivation. HSPs act as chaperones to other cellular proteins (called client proteins) and facilitate their proper folding and repair, and aid in the refolding of misfolded client proteins. There are several known families of HSPs, each having its own set of client proteins. The Hsp90 family is one of the most abundant HSP families, accounting for about 1-2% of proteins in a cell that is not under stress and increasing to about 4-6% in a cell under stress. Inhibition of Hsp90 results in degradation of its client proteins via the ubiquitin proteasome pathway. Unlike other chaperone proteins, the client proteins of Hsp90 are mostly protein kinases or transcription factors involved in signal transduction, and a number of its client proteins have been shown to be involved in the progression of cancer.

### SUMMARY OF THE INVENTION

It is found that certain triazolone Hsp90 inhibitors are surprisingly effective in treating a subject with cancer, where the cancer is mediated through dysregulated, aberrant, or defective JAK/STAT signaling. The present invention provides a method of treating or preventing cancer in a subject in need thereof, where the cancer is mediated through dysregulated, aberrant, or defective JAK/STAT signaling, comprising: determining the level of JAK/STAT signaling in a sample derived from the subject; and administering to the subject an effective amount of a triazolone compound of formulae (I) or (Ia), or a compound in Table 1 or Table 2, wherein the presence of dysregulated, or aberrant, or defective JAK/STAT signaling is indicated.

In one embodiment, the method also includes improving the efficacy of a triazolone compound in the treatment of a subject with cancer, comprising (a) determining the level of JAK/STAT signaling in a sample derived from the subject; and (b) administering to the subject an effective amount of the triazolone compound represented by the structural formulae (I) or (Ia) as defined above or a compound in Table 1 or Table 2, wherein the presence of dysregulated or aberrant or defective JAK/STAT signaling level is indicated.

In one embodiment, the method also includes inhibiting or treating cancer or tumor cells in a subject with cancer, comprising (a) determining the level of JAK/STAT signaling in sample from the subject; and (b) administering to the subject an effective amount of a triazolone compound represented by formula (I) or (Ia), or a compound in Table 1 or Table 2, or a tautomer or a pharmaceutically acceptable salt thereof, wherein the presence of dysregulated or aberrant JAK/STAT signaling level is indicated.

In one embodiment, the triazolone compound may be 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof. In one embodiment, the triazolone compound may be 5-hydroxy-4-(5-hydroxy-4-(1-methyl-1H-indol-5-yl)-4H-1,2,4-triazol-3-yl)-2-isopropylphenyl dihydrogen phosphate dihydrogen phosphate, or a tautomer, or a pharmaceutically acceptable salt thereof.

In any one of these embodiments, the cancer may be lung cancer, breast cancer, hematological neoplasms, gastrointestinal stromal tumor, pancreatic cancer, prostate cancer, leukemia, myeloproliferative neoplasms, solid cancer, or non-small cell lung cancer.

In any one of these embodiments, the dysregulated, aberrant, or defective JAK/STAT signaling may be mediated through dysregulated JAK protein. In another embodiment, the dysregulated JAK protein may be mediated through one or more mutations of JAK2 protein.

In any one of these embodiments, the dysregulated, aberrant, or defective JAK/STAT signaling may be mediated through dysregulated STAT3 protein activity. In any one of these embodiments, the dysregulated, aberrant, or defective JAK/STAT signaling may be mediated through dysregulated STAT5 protein activity.

In any one of these embodiments, the dysregulated, aberrant, or defective JAK/STAT signaling may be mediated through dysregulated, aberrant, or defective JAK2/STAT3 signaling.

In any one of these embodiments, the dysregulated, aberrant, or defective JAK/STAT signaling may be mediated through dysregulated, aberrant, or defective JAK2/STAT5 signaling.

In one embodiment, the method includes administering to a subject with cancer an effective amount of the triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, wherein the presence of dysregulated, aberrant, or defective JAK/STAT signaling in the subject is indicated.

In another embodiment, the method includes administering to a subject with cancer an effective amount of the triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, wherein the presence of dysregulated, aberrant, or defective JAK/STAT signaling in the subject is indicated.

Another embodiment includes the use of an Hsp90 inhibitor described herein for the manufacture of a medicament for treating JAK/STAT signaling-mediated cancer, or for treating cancer wherein the cancer is mediated through or associated with dysregulated, aberrant or defective JAK/STAT signaling. In yet another embodiment, the method includes using a triazolone Hsp90 inhibitor described herein with another therapeutic agent in treating JAK/STAT signaling-mediated cancer.

### BRIEF DESCRIPTION OF THE FIGURES

FIG. 1 shows the effects of ganetespib (also called STA-9090, or compound 1) on tumor cell viability. SET-2, HEL92.1.7, MV4-11, NCI-H1975 and DU145 cells were treated with ganetespib or 17-AAG over a broad dose range (0.0001 to 1 μM) for 72 h and cell viability assessed by Alamar blue staining.

FIG. 2 compares the more durable ganetespib durable inhibition of JAK/STAT signaling to P6. HEL92.1.7 cells were cultured in the presence of 250 nM Ganetespib or 1000 nM P6 and harvested at the indicated time points. The levels of JAK2, phosphorylated and total STAT3 and STAT5, phospho-AKT, phospho-ERK and GAPDH were determined by western blot.

FIG. 3 demonstrates the higher potency of ganetespib compared to 17-AAG. SET-2 cells were dosed with the indicated concentrations of ganetespib or 17-AAG for 24 h and analyzed to determine JAK/STAT protein and target levels using the antibodies indicated.

FIG. 4 shows the inhibition of JAK2/STAT signaling by ganetespib in solid tumors (client protein down-regulation in NSCLC). NCI-H1975 cells were dosed with the indicated concentrations of ganetespib for 24 h and their cell lysates analyzed to determine JAK/STAT and Hsp90 client protein levels using the antibodies indicated.

FIG. 5 shows ganetespib blocking IL-6 induced and constitutive STAT3 activity in NSCLC cells. HCC827 lung cancer cells were treated with increasing concentrations of ganetespib or P6 for 24 h followed by a 15 min stimulation with or without 50 ng/ml human recombinant IL-6. The levels of JAK2, total and phospho-STAT3, and PIM2 were analyzed by western blot. GAPDH is included as a loading control.

FIG. 6 shows client protein degradation in prostate cancer cells. DU145 cells were dosed with graded concentrations of ganetespib for 24 h and cell lysates subject to western blot to determine JAK/STAT and target protein levels using the antibodies indicated.

FIG. 7 shows that functional Hsp90 was required for JAK2, but not JAK1, stability in DU145 cells. DU145 cells were treated with DMSO (control, C), 15 nM, 60 nM or 240 nM ganetespib for either 24 or 48 h and lysates probed by western blot with the indicated antibodies.

FIG. 8 shows that ganetespib inhibits JAK/STAT target and cell cycle gene expression. Comparative effects of ganetespib and P6 on HEL92.1.7 tumor cell viability. HEL92.1.7 cells were treated with ganetespib or P6 over a broad dose range (0.0001 to 10 μM) for 72 h and cell viability assessed by Alamar blue.

FIG. 9 shows temporal and dose-dependent effects on JAK/STAT targets by ganetespib and P6. HEL92.1.7 cells were treated with ganetespib at 25 nM or 250 nM or P6 at 100 nM or 1000 nM for 4 and 24 h and cell lysates subject to western blot to determine JAK2/STAT and target protein levels using the indicated antibodies.

FIG. 10 shows inhibition of JAK2 activity by P6 (1000 nM) or destabilization of JAK2 expression by ganetespib (250 nM) blocked STAT-target gene transcription. Inhibition of Hsp90 by ganetespib resulted in the up-regulation of heat shock protein genes. Values represent cycle threshold (Ct), normalized to HPRT.

FIG. 11 shows HEL92.1.7 cells dosing with ganetespib (250 nM) or P6 (1000 nM) for 48 h. Cells were harvested at the indicated time points and the levels of total and phospho-Cdk1, phospho-Chk2 and GAPDH analyzed by western blot.

FIG. 12 illustrates the effects of ganetespib on JAK/STAT and cell cycle protein expression. HEL92.1.7 cells were treated with 100 nM ganetespib and subject to western blot to determine the levels of Cdk1, cyclin B1, cyclin A1, JAK2, total and phospho-STAT3, and GAPDH at hourly intervals over an 11 h time course.

FIG. 13 shows the results of MCF-7, GIST882, HPAF and DU145 cells dosed with graded concentrations of ganetespib for 24 h followed by Western blot analysis using the antibodies described hereinbelow.

FIG. 14 shows the in vivo efficacy of ganetespib in a leukemia survival model expressing activated JAK2V617F by Kaplan-Meier analysis of overall survival in a leukemia model established by i.v. injection of HEL92.1.7 cells into SCID mice, which resulted in the development of disseminated disease. Beginning 1 day after tumor cell implantation, ganetespib was i.v. dosed at its HNSTD (25 mg/kg) on a five-times per week schedule for 3 weeks through day 19 (n=−21-10/group). *P<0.0001; 2-sided log-rank test.

FIG. 15 shows that ganetespib was well tolerated in the HEL92.1.7 disseminated leukemia model. Cumulative average body weights showed minimal effects over the 3 week dosing period. Points represent the means and the error bars are the s.e.m.

FIG. 16 shows ganetespib efficacy and pharmacodynamics in an in vivo leukemia model with constitutively activated STATS signaling as SCID mice were subcutaneously implanted with MV4-11 acute myeloid leukemia cells. Mice bearing established MV4-11 xenografts (100-200 mm3, n=8 mice/group) were i.v. dosed (arrowheads) with ganetespib at either 25 or 150 mg/kg once weekly for 3 weeks, or at the HNSTD of 25 mg/kg five-times per week, as indicated. % T/C values are indicated to the right of each growth curve and the error bars are the s.e.m.

FIG. 17 shows that ganetespib inhibits STAT-5 phosphorylation and Cdk1 expression in tumor xenografts in SCID mice. SCID mice bearing MV4-11 tumors (n=4 mice/group) were treated with vehicle or ganetespib at either 25 mg/kg or 150 mg/kg at the indicated time points between 6 h and 144 h (6 days). Tumors were resected and the levels of p-STATS, Cdk1, Hsp70 and GAPDH were determined by western blot.

### DETAILED DESCRIPTION OF THE INVENTION

### Definitions

Unless otherwise specified, the below terms used herein are defined as follows:

As used herein, the term “alkyl” means a saturated, straight chain or branched, non-cyclic hydrocarbon having from 1 to 10 carbon atoms. Representative straight chain alkyls include methyl, ethyl, n-propyl, n-butyl, n-pentyl, n-hexyl, n-heptyl, n-octyl, n-nonyl and n-decyl; while representative branched alkyls include isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimtheylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl, and the like. The term “(C₁-C₆)alkyl” means a saturated, straight chain or branched, non-cyclic hydrocarbon having from 1 to 6 carbon atoms. Alkyl groups included in compounds described herein may be optionally substituted with one or more substituents.

As used herein, the term “alkenyl” means a straight chain or branched, non-cyclic hydrocarbon having from 2 to 10 carbon atoms and having at least one carbon-carbon double bond. Representative straight chain and branched (C₂-C₁₀)alkenyls include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl, 3-decenyl, and the like. Alkenyl groups included in compounds described herein may be optionally substituted with one or more substituents.

As used herein, the term “alkynyl” means a straight chain or branched, non-cyclic hydrocarbon having from 2 to 10 carbon atoms and having at least one carbon-carbon triple bond. Representative straight chain and branched alkynyls include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl, 9-decynyl, and the like. Alkynyl groups included in compounds described herein may be optionally substituted with one or more substituents.

As used herein, the term “cycloalkyl” means a saturated, mono- or polycyclic, non-aromatic hydrocarbon having from 3 to 20 carbon atoms. Representative cycloalkyls include cyclopropyl, 1-methylcyclopropyl, cyclobutyl, cyclopentyl, cyclohexyl, cycloheptyl, cyclooctyl, cyclononyl, cyclodecyl, octahydropentalenyl, and the like. Cycloalkyl groups included in compounds described herein may be optionally substituted with one or more substituents.

As used herein, the term “cycloalkenyl” means a mono- or polycyclic, non-aromatic hydrocarbon having at least one carbon-carbon double bond in the cyclic system and having from 3 to 20 carbon atoms. Representative cycloalkenyls include cyclopentenyl, cyclopentadienyl, cyclohexenyl, cyclohexadienyl, cycloheptenyl, cycloheptadienyl, cycloheptatrienyl, cyclooctenyl, cyclooctadienyl, cyclooctatrienyl, cyclooctatetraenyl, cyclononenyl, cyclononadienyl, cyclodecenyl, cyclodecadienyl, 1,2,3,4,5,8-hexahydronaphthalenyl, and the like. Cycloalkenyl groups included in compounds described herein may be optionally substituted with one or more substituents.

As used herein, the term “alkylene” refers to an alkyl group that has two points of attachment. The term “(C₁-C₆)alkylene” refers to an alkylene group that has from one to six carbon atoms. Straight chain (C₁-C₆)alkylene groups are preferred. Non-limiting examples of alkylene groups include methylene (—CH₂—), ethylene (—CH₂CH₂—), n-propylene (—CH₂CH₂CH₂—), isopropylene (—CH₂CH(CH₃)—), and the like. Alkylene groups included in compounds described herein may be optionally substituted with one or more substituents.

As used herein, the term “lower” refers to a group having up to four atoms. For example, a “lower alkyl” refers to an alkyl radical having from 1 to 4 carbon atoms, “lower alkoxy” refers to “—O—(C₁-C₄)alkyl and a “lower alkenyl” or “lower alkynyl” refers to an alkenyl or alkynyl radical having from 2 to 4 carbon atoms.

As used herein, the term “haloalkyl” means an alkyl group, in which one or more, including all, the hydrogen radicals are replaced by a halo group(s), wherein each halo group is independently selected from —F, —Cl, —Br, and —I. For example, the term “halomethyl” means a methyl in which one to three hydrogen radical(s) have been replaced by a halo group. Representative haloalkyl groups include trifluoromethyl, bromomethyl, 1,2-dichloroethyl, 4-iodobutyl, 2-fluoropentyl, and the like.

As used herein, an “alkoxy” is an alkyl group which is attached to another moiety via an oxygen linker. Alkoxy groups included in compounds described herein may be optionally substituted with one or more substituents.

As used herein, a “haloalkoxy” is a haloalkyl group which is attached to another moiety via an oxygen linker.

As used herein, the term an “aromatic ring” or “aryl” means a mono- or polycyclic hydrocarbon, containing from 6 to 15 carbon atoms, in which at least one ring is aromatic. Examples of suitable aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. Aryl groups included in compounds described herein may be optionally substituted with one or more substituents. In one embodiment, the aryl group is a monocyclic ring, wherein the ring comprises 6 carbon atoms, referred to herein as “(C₆)aryl.”

As used herein, the term “aralkyl” means an aryl group that is attached to another group by a (C₁-C₆)alkylene group. Representative aralkyl groups include benzyl, 2-phenyl-ethyl, naphth-3-yl-methyl and the like. Aralkyl groups included in compounds described herein may be optionally substituted with one or more substituents.

As used herein, the term “heterocyclyl” means a monocyclic or a polycyclic, saturated or unsaturated, non-aromatic ring or ring system which typically contains 5- to 20-members and at least one heteroatom. A heterocyclic ring system can contain saturated ring(s) or unsaturated non-aromatic ring(s), or a mixture thereof. A 3- to 10-membered heterocycle can contain up to 5 heteroatoms, and a 7- to 20-membered heterocycle can contain up to 7 heteroatoms. Typically, a heterocycle has at least one carbon atom ring member. Each heteroatom is independently selected from nitrogen, which can be oxidized (e.g., N(O)) or quaternized, oxygen and sulfur, including sulfoxide and sulfone. The heterocycle may be attached via any heteroatom or carbon atom. Representative heterocycles include morpholinyl, thiomorpholinyl, pyrrolidinonyl, pyrrolidinyl, piperidinyl, piperazinyl, hydantoinyl, valerolactamyl, oxiranyl, oxetanyl, tetrahydrofuranyl, tetrahydropyranyl, tetrahydropyrindinyl, tetrahydropyrimidinyl, tetrahydrothiophenyl, tetrahydrothiopyranyl, and the like. A heteroatom may be substituted with a protecting group known to those of ordinary skill in the art, for example, a nitrogen atom may be substituted with a tert-butoxycarbonyl group. Furthermore, the heterocyclyl included in compounds described herein may be optionally substituted with one or more substituents. Only stable isomers of such substituted heterocyclic groups are contemplated in this definition.

As used herein, the term “heteroaromatic”, “heteroaryl”, or like terms, means a monocyclic or a polycyclic, unsaturated radical containing at least one heteroatom, in which at least one ring is aromatic. Polycyclic heteroaryl rings must contain at least one heteroatom, but not all rings of a polycyclic heteroaryl moiety must contain heteroatoms. Each heteroatom is independently selected from nitrogen, which can be oxidized (e.g., N(O)) or quaternized, oxygen and sulfur, including sulfoxide and sulfone. Representative heteroaryl groups include pyridyl, 1-oxo-pyridyl, furanyl, benzo[1,3]dioxolyl, benzo[1,4]dioxinyl, thienyl, pyrrolyl, oxazolyl, imidazolyl, thiazolyl, a isoxazolyl, quinolinyl, pyrazolyl, isothiazolyl, pyridazinyl, pyrimidinyl, pyrazinyl, a triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidinyl, pyrazolo[3,4]pyrimidinyl, imidazo[1,2-a]pyridyl, and benzothienyl. In one embodiment, the heteroaromatic ring is selected from 5-8 membered monocyclic heteroaryl rings. The point of attachment of a heteroaromatic or heteroaryl ring may be at either a carbon atom or a heteroatom. Heteroaryl groups included in compounds described herein may be optionally substituted with one or more substituents. As used herein, the term “(C₆)heteroaryl” means an heteroaromatic ring of 5 members, wherein at least one carbon atom of the ring is replaced with a heteroatom, such as, for example, oxygen, sulfur or nitrogen. Representative (C₆)heteroaryls include furanyl, thienyl, pyrrolyl, oxazolyl, imidazolyl, thiazolyl, isoxazolyl, pyrazolyl, isothiazolyl, pyrazinyl, triazolyl, thiadiazolyl, and the like. As used herein, the term “(C₆)heteroaryl” means an aromatic heterocyclic ring of 6 members, wherein at least one carbon atom of the ring is replaced with a heteroatom such as, for example, oxygen, nitrogen or sulfur. Representative (C₆)heteroaryls include pyridyl, pyridazinyl, pyrazinyl, triazinyl, tetrazinyl, and the like.

As used herein, the term “heteroaralkyl” means a heteroaryl group that is attached to another group by a (C₁-C₆)alkylene. Representative heteroaralkyls include 2-(pyridin-4-yl)-propyl, 2-(thien-3-yl)-ethyl, imidazol-4-yl-methyl, and the like. Heteroaralkyl groups included in compounds described herein may be optionally substituted with one or more substituents.

As used herein, the term “halogen” or “halo” means —F, —Cl, —Br or —I.

As used herein the term “heteroalkyl” means a straight or branched alkyl group wherein one or more of the internal carbon atoms in the chain is replaced by a heteroatom. For example, a heteroalkyl is represented by the formula —[CH₂]ₓ—Z—[CH₂]_{y}[CH₃], wherein x is a positive integer and y is zero or a positive integer, Z is O, NR, S, S(O), or S(O)₂, and wherein replacement of the carbon atom does not result in a unstable compound. Heteroalkyl groups included in compounds described herein may be optionally substituted with one or more substituents.

Suitable substituents for an alkyl, alkylene, alkenyl, alkynyl, cycloalkyl, cycloalkenyl, heterocyclyl, aryl, aralkyl, heteroaryl, and heteroaralkyl groups include are those substituents which form a stable compound described herein without significantly adversely affecting the reactivity or biological activity of the compound described herein. Examples of substituents for an alkyl, alkylene, alkenyl, alkynyl, cycloalkyl, cycloalkenyl, heterocyclyl, aryl, aralkyl, heteroaryl, and heteroaralkyl include an alkyl, alkenyl, alkynyl, cycloalkyl, cycloalkenyl, heterocyclyl, aryl, heteroaryl, aralkyl, heteraralkyl, heteroalkyl, alkoxy, (each of which can be optionally and independently substituted), —C(O)NR²⁸R²⁹, —C(S)NR²⁸R²⁹, —C(NR³²)NR²⁸R²⁹, —NR³³C(O)R³¹, —NR³³C(S)R³¹, —NR³³C(NR³²)R³¹, halo, —OR³³, cyano, nitro, —C(O)R³³, —C(S)R³³, —C(NR³²)R³³, —NR²⁸R²⁹, —C(O)OR³³, —C(S)OR³³, —C(NR³²)OR³³, —OC(O)R³³, —OC(S)R³³, —OC(NR³²)R³³, —NR³⁰C(O)NR²⁸R²⁹, —NR³³C(S)NR²⁸R²⁹, —NR³³C(NR³²)NR²⁸R²⁹, —OC(O)NR²⁸R²⁹, —OC(S)NR²⁸R²⁹, —OC(NR³²)NR²⁸R²⁹, —NR³³C(O)OR³¹, —NR³³C(S)OR³¹, —NR³³C(NR³²)OR³¹, —S(O)ₖR³³, —OS(O)ₖR³³, —NR³³S(O)ₖR³³, —S(O)ₖNR²⁸R²⁹, —OS(O)ₖNR²⁸R²⁹—NR³³S(O)ₖNR²⁸R²⁹, guanidino, —C(O)SR³¹, —C(S)SR³¹, —C(NR³²)SR³¹, —OC(O)OR³¹, —OC(S)OR³¹, —OC(NR³²)OR³¹, —SC(O)R³³, —SC(O)OR³¹, —SC(NR³²)OR³¹, —SC(S)R³³, —SC(S)OR³¹, —SC(O)NR²⁸R²⁹, —SC(NR³²)NR²⁸R²⁹, —SC(S)NR²⁸R²⁹, —SC(NR³²)R³³, —OS(O)ₖOR³¹, —S(O)ₖOR³¹, —NR³⁰S(O)ₖOR³¹, —SS(O)ₖR³³, —SS(O)ₖOR³¹, —SS(O)ₖNR²⁸R²⁹, —OP(O)(OR³¹)₂, or —SP(O)(OR³¹)₂. In addition, any saturated portion of an alkyl, cycloalkyl, alkylene, heterocyclyl, alkenyl, cycloalkenyl, alkynyl, aralkyl and heteroaralkyl groups, may also be substituted with ═O, ═S, or ═N—R³².

Each R²⁸, R²⁹, and R³⁰ is independently H, alkyl, alkenyl, alkynyl, cycloalkyl, cycloalkenyl, heterocyclyl, aryl, heteroaryl, aralkyl, or heteraralkyl, wherein each alkyl, alkenyl, alkynyl, cycloalkyl, cycloalkenyl, heterocyclyl, aryl, heteroaryl, aralkyl, or heteroalkyl represented by R²⁸ or R²⁹, or R³⁰ is optionally and independently substituted.

Each R³¹ and R³³ is independently H, alkyl, alkenyl, alkynyl, cycloalkyl, cycloalkenyl, heterocyclyl, aryl, heteroaryl, aralkyl, or heteraralkyl, wherein each alkyl, alkenyl, alkynyl, cycloalkyl, cycloalkenyl, heterocyclyl, aryl, heteroaryl, aralkyl, and heteraralkyl represented by R³¹ or R³³ is optionally and independently unsubstituted.

Each R³² is independently H, alkyl, alkenyl, alkynyl, cycloalkyl, cycloalkenyl, heterocyclyl, aryl, heteroaryl, aralkyl, heteraralkyl, —C(O)R³³, —C(O)NR²⁸R²⁹, —S(O)ₖR³³ or —S(O)ₖNR²⁸R²⁹, wherein each alkyl, alkenyl, alkynyl, cycloalkyl, cycloalkenyl, heterocyclyl, aryl, heteroaryl, aralkyl and heteraralkyl represented by R³² is optionally and independently substituted.

The variable k is 0, 1 or 2.

When a heterocyclyl, heteroaryl or heteroaralkyl group contains a nitrogen atom, it may be substituted or unsubstituted. When a nitrogen atom in the aromatic ring of a heteroaryl group has a substituent, the nitrogen may be oxidized or a quaternary nitrogen.

As used herein, the term “compound(s) of this invention”, “triazolone compound”, or similar terms refers to a compound of any one of formulae (I) or (Ia) or a compound in Table 1 or 2, or a pharmaceutically acceptable salt thereof.

The compounds described herein are defined by their chemical structures and/or chemical names. Where a compound is referred to by both a chemical structure and a chemical name, and the chemical structure and chemical name conflict, the chemical structure is determinative of the compound's identity.

Only those choices and combinations of substituents that result in a stable structure are contemplated. Such choices and combinations will be apparent to those of ordinary skill in the art and may be determined without undue experimentation.

The Janus kinase/signal transducers and activators of transcription (JAK/STAT) pathway is one of a handful of pleiotropic cascades used to transduce a multitude of signals for development and homeostasis in animals, from humans to flies. In mammals, the JAK/STAT pathway is the principal signaling mechanism for a wide array of cytokines and growth factors. JAK activation stimulates cell proliferation, differentiation, cell migration and apoptosis. These cellular events are critical to hematopoiesis, immune development, mammary gland development and lactation, adipogenesis, sexually dimorphic growth and other processes. Predictably, mutations that reduce JAK/STAT pathway activity affect these processes. See, e.g., Igaz et al (2001). Biological and clinical significance of the JAK-STAT pathway; lessons from knockout mice. Inflamm. Res. 50, 435-441; O'Shea et al (2002). Cytokine signaling in 2002: new surprises in the Jak/Stat pathway. Cell, 109 Suppl. S121-S131. In mammals, the JAK family comprises four members: JAK1, JAK2, JAK 3 and Tyk2. The STAT gene family consists of seven proteins (STAT1, STAT2, STAT3, STAT4, STAT5a, STAT5b and STAT6).

The JAK proteins are established Hsp90 client proteins. Dysregulated, or aberrant, or defective JAK signaling can result in the constitutive activation of Signal Transducers and Activators of Transcription (STAT) transcription factors leading to oncogenic growth. The terms of “dysregulated”, “aberrant”, or “defective” used herein are interchangeable, all meaning deviations from a normal situation, particularly herein from normal JAK/STAT signaling. Inappropriate activation of JAK signaling underlies cell proliferation and survival in a variety of solid tumors, including lung, breast and prostate cancer, as well as in hematological neoplasms. JAK2 is a ubiquitously expressed member of the JAK family of nonreceptor tyrosine kinases which function to mediate signaling downstream of cytokine and growth factor receptors. In particular, an activating point mutation in JAK2 (JAK2V617F) has been described with high frequency in chronic myeloproliferative disorders (MPD) and constitutive JAK2 activation caused by chromosomal translocations has been reported in various types of leukemia. Activated cytokine-JAK complexes recruit and phosphorylate effector molecules including STAT proteins. STAT proteins mediate a wide range of biological processes, including cell growth, differentiation, apoptosis, inflammation and immune response. Two STATs in particular, STAT3 and STAT5, represent the major substrates for JAK2 that govern myelopoeisis and can contribute to cellular transformation. Their persistent activation has been linked to increased tumor cell proliferation, survival, metastasis and tumor-promoting inflammation in both solid and hematological tumors.

Persistent JAK/STAT activation is oncogenic and characteristic of many human malignancies and thereby provides an attractive point of intervention for molecularly targeted therapeutics. It has been found that triazolone compounds disclosed herein, e.g., ganetespib, have significant antitumor activity in an array of JAK/STAT-driven cancers and could abrogate aberrant signaling through multiple mechanisms. Without being bound by any theory, these triazolone are thought to compounds effectively target the upstream regulator JAK2, including the constitutively active JAK2V617F mutant, for degradation in a range of hematological and solid tumor types with subsequent prolonged loss of STAT3 and STAT5 signaling. These findings indicate the possible pathogenic role of STAT signaling in tumorigenesis. As described in various examples herein below, the sustained inhibition of the JAK2/STAT signaling axis achieved by ganetespib was more effective than that seen with the pan-JAK inhibitor P6, and ganetespib was found to be significantly more potent than the first generation Hsp90 inhibitor 17-AAG.

While JAK2 mutation is a common means to stimulate oncogenic STAT activity, perturbations in other signaling networks, such as those mediated by EGFR, IL-6/IL-6R or FLT3, can also contribute to activated STAT signaling in cancer cells. Hsp90 inhibition effectively disrupts these as well, with the triazolone compounds described herein potently degrading EGFR and blocking both IL-6- and FLT3-mediated activation of STAT proteins. Thus, while these compounds directly impose their pharmacological effects on Hsp90, the downstream consequences involve a substantial array of client proteins and biochemical pathways. Hsp90 inhibition by these triazolone compounds may be viewed as a multi-nodal modality, rather than a target-specific therapeutic approach, such as that engendered by a JAK2 or other kinase inhibitor.

As described in the examples hereinbelow, both ganetespib and P6 alter a common set of JAK/STAT targets, but only ganetespib treatment exerted concomitant effects on the cell cycle regulatory machinery. Exposure to ganetespib in leukemic cells resulted in G1 and G2/M arrest, in part through the degradation of Cdk1 and atypical accumulation of cyclins A1 and B1. S phase was also abrogated. Several components of the centrosome and spindle were affected at the transcriptional level by ganetespib, in agreement with the findings that these components are synthesized in S phase and that Hsp90 is essential for centrosome assembly. This was a general response in all cells studied, as similar combinatorial effects were observed on JAK/STAT inhibition with loss of cyclin-dependent kinase activity in AML, breast, gastrointestinal stromal, pancreatic and prostate tumor types.

These triazolone compounds show potent in vivo activity. In mice with established MV4-11 (STAT5-driven) xenografts, ganetespib significantly inhibits tumor growth in a dose-dependent manner. Moreover, a daily dosing schedule of ganetespib resulted in significant tumor regression during drug administration. In this model, tumor growth reappears about a week after the drug treatment was stopped (for the high dose, ix/week cohort). Pharmacodynamic analysis showed that these tumor responses correlated with the degree and duration of STAT5 and Cdk1 protein loss induced by the varying dosing regimens. The tight linkage of STAT5 down-regulation with inhibition of tumor growth soon after drug administration at either dose (6 hours) indicated the quick response of this signaling pathway to the drug administration. At the 150 mg/kg dose of ganetespib, STAT5 signaling, but not Cdk1 expression, returned by six days.

The sustained loss of Cdk1 and other cell cycle proteins presumably maintained the cell cycle arrest and prevented growth from re-occurring between doses on the weekly schedule, even in the presence of the re-emergent STAT5 activity. Similarly, Cdk1 expression was suppressed longer in comparison to STAT5 at the 25 mg/kg dose of ganetespib, and was likely to account for the potent activity of ganetespib on the more frequent 5×/week regimen. It was also found that ganetespib administration on either schedule was sufficient to abolish both survival and cell growth signals long enough to prevent tumor growth. Because ganetespib administration is believed to lead to the loss of even more client proteins, its potent antitumor activity likely reflects its combined impact on these additional target proteins as well.

In a system that more accurately mimics the pathology of leukemic diseases, the efficacy of ganetespib was also evaluated in a disseminated disease model using HEL92.1.7 cells. Ganetespib effectively increased survival in this orthotopic model, more than doubling the median survival time of the mice. Prolonged survival was associated with dramatically reduced tumor burden in the bone marrow, as evidenced by significantly decreased infiltration of human leukemic cells and reduced spinal column metastases. Collectively, these data were consistent with a direct effect of ganetespib on leukemic cell growth in vivo and demonstrates the potential therapeutic utility of this compound for JAK2V617F-driven malignancies.

These triazolone compounds, e.g., ganetespib, are shown herein to have potent in vitro and in vivo activity in tumor cells harboring constitutively active JAK/STAT signaling. Through its concomitant effects on both oncogenic signaling and cell cycle progression, ganetespib is superior to both 17-AAG and the pan-JAK inhibitor P6 in terms of potency, duration of response, and preclinical efficacy.

As used herein, the terms “subject”, “patient” and “mammal” are used interchangeably. The terms “subject” and “patient” refer to an animal (e.g., a bird such as a chicken, quail or turkey, or a mammal), preferably a mammal including a non-primate (e.g., a cow, pig, horse, sheep, rabbit, guinea pig, rat, cat, dog, and mouse) and a primate (e.g., a monkey, chimpanzee and a human), and more preferably a human. In one embodiment, the subject is a non-human animal such as a farm animal (e.g., a horse, cow, pig or sheep), or a pet (e.g., a dog, cat, guinea pig or rabbit). In a preferred embodiment, the subject is a human.

As used herein, the term “pharmaceutically acceptable salt” refers to a salt prepared from a compound of any one of formulae (I) or (Ia) or a compound in Table 1 or Table 2 having an acidic functional group, such as a carboxylic acid functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl, N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N,N,-di-lower alkyl-N-(hydroxy lower alkyl)-amines, such as N,N-dimethyl-N-(2-hydroxyethyl)amine, or tri-(2-hydroxyethyl)amine; N-methyl-D-glucamine; and amino acids such as arginine, lysine, and the like. The term “pharmaceutically acceptable salt” also refers to a salt prepared from a compound of any one of formulae (I) or (Ia) or a compound in Table 1 or Table 2 having a basic functional group, such as an amine functional group, and a pharmaceutically acceptable inorganic or organic acid. Suitable acids include, but are not limited to, hydrogen sulfate, citric acid, acetic acid, oxalic acid, hydrochloric acid (HCl), hydrogen bromide (HBr), hydrogen iodide (HI), nitric acid, hydrogen bisulfide, phosphoric acid, isonicotinic acid, oleic acid, tannic acid, pantothenic acid, saccharic acid, lactic acid, salicylic acid, tartaric acid, bitartratic acid, ascorbic acid, succinic acid, maleic acid, besylic acid, fumaric acid, gluconic acid, glucaronic acid, formic acid, benzoic acid, glutamic acid, methanesulfonic acid, ethanesulfonic acid, benzenesulfonic acid, pamoic acid and p-toluenesulfonic acid.

A pharmaceutically acceptable carrier may contain inert ingredients which do not unduly inhibit the biological activity of the compound(s). The pharmaceutically acceptable carriers should be biocompatible, i.e., non-toxic, non-inflammatory, non-immunogenic and devoid of other undesired reactions upon the administration to a subject. Standard pharmaceutical formulation techniques can be employed, such as those described in REMINGTON, J. P., REMINGTON'S PHARMACEUTICAL SCIENCES (Mack Pub. Co., 17^{th} ed., 1985). Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate, and the like. Methods for encapsulating compositions, such as in a coating of hard gelatin or cyclodextran, are known in the art. See BAKER, ET AL., CONTROLLED RELEASE OF BIOLOGICAL ACTIVE AGENTS, (John Wiley and Sons, 1986).

As used herein, the term “effective amount” refers to an amount of a compound described herein which is sufficient to reduce or ameliorate the severity, duration, progression, or onset of a disease or disorder, delay onset of a disease or disorder, retard or halt the advancement of a disease or disorder, cause the regression of a disease or disorder, prevent or delay the recurrence, development, onset or progression of a symptom associated with a disease or disorder, or enhance or improve the therapeutic effect(s) of another therapy. The precise amount of compound administered to a subject will depend on the mode of administration, the type and severity of the disease or condition and on the characteristics of the subject, such as general health, age, sex, body weight and tolerance to drugs. For example, for a proliferative disease or disorder, determination of an effective amount will also depend on the degree, severity and type of cell proliferation. The skilled artisan will be able to determine appropriate dosages depending on these and other factors. When co-administered with other therapeutic agents, e.g., when co-administered with an anti-cancer agent, an “effective amount” of any additional therapeutic agent(s) will depend on the type of drug used. Suitable dosages are known for approved therapeutic agents and can be adjusted by the skilled artisan according to the condition of the subject, the type of condition(s) being treated and the amount of a compound of the invention being used. In cases where no amount is expressly noted, an effective amount should be assumed.

The dosage of a therapeutic agent other than a compound described herein, which has been or is currently being used to treat, manage, or ameliorate cancer, or one or more symptoms thereof, can be used in the methods described herein. Preferably, the dosage of each individual therapeutic agent used in the therapy is lower than the dose of an individual therapeutic agent when given independently to treat, manage, or ameliorate a disease or disorder, or one or more symptoms thereof. The recommended dosages of therapeutic agents currently used for the treatment, management, or amelioration of a disease or disorder, or one or more symptoms thereof, can obtained from any reference in the art. See, e.g., GOODMAN & GILMAN'S THE PHARMACOLOGICAL BASIS OF BASIS OF THERAPEUTICS 9^{TH} ED, (Hardman, et al., Eds., NY: McGraw-Hill (1996)); PHYSICIAN'S DESK REFERENCE 57^{TH} ED. (Medical Economics Co., Inc., Montvale, N.J. (2003)).

As used herein, the terms “treat”, “treatment” and “treating” refer to the reduction or amelioration of the progression, severity and/or duration of a disease or disorder, delay of the onset of a disease or disorder, or the amelioration of one or more symptoms (preferably, one or more discernible symptoms) of a disease or disorder, resulting from the administration of one or more therapies (e.g., one or more therapeutic agents such as a compound of the invention). The terms “treat”, “treatment” and “treating” also encompass the reduction of the risk of developing a disease or disorder, and the delay or inhibition of the recurrence of a disease or disorder. In specific embodiments, the terms “treat”, “treatment” and “treating” refer to the amelioration of at least one measurable physical parameter of a disease or disorder, such as growth of a tumor, not necessarily discernible by the patient. In other embodiments the terms “treat”, “treatment” and “treating” refer to the inhibition of the progression of a disease or disorder, e.g., cancer, either physically by the stabilization of a discernible symptom, physiologically by the stabilization of a physical parameter, or both. In another embodiment, the terms “treat”, “treatment” and “treating” of a proliferative disease or disorder refers to the reduction or stabilization of tumor size or cancerous cell count, and/or delay of tumor formation.

As used herein, the terms “therapeutic agent” and “therapeutic agents” refer to any agent(s) that can be used in the treatment of a disease or disorder, e.g. cancer, or one or more symptoms thereof. In certain embodiments, the term “therapeutic agent” refers to a compound described herein. In certain other embodiments, the term “therapeutic agent” does not refer to a compound described herein. Preferably, a therapeutic agent is an agent that is known to be useful for, or has been or is currently being used for the treatment of a disease or disorder, e.g., cancer, or one or more symptoms thereof.

As used herein, the term “synergistic” refers to a combination of a compound described herein and another therapeutic agent, which, when taken together, is more effective than the additive effects of the individual therapies. A synergistic effect of a combination of therapies (e.g., a combination of therapeutic agents) permits the use of lower dosages of one or more of the therapeutic agent(s) and/or less frequent administration of the agent(s) to a subject with a disease or disorder, e.g., cancer. The ability to utilize lower dosage of one or more therapeutic agent and/or to administer the therapeutic agent less frequently reduces the toxicity associated with the administration of the agent to a subject without reducing the efficacy of the therapy in the treatment of a disease or disorder. In addition, a synergistic effect can result in improved efficacy of agents in the prevention, management or treatment of a disease or disorder, e.g. cancer. Finally, a synergistic effect of a combination of therapies may avoid or reduce adverse or unwanted side effects associated with the use of either therapeutic agent alone.

As used herein, the term “in combination” refers to the use of more than one therapeutic agent. The use of the term “in combination” does not restrict the order in which the therapeutic agents are administered to a subject with cancer. A first therapeutic agent, such as a compound described herein, can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent, such as an anti-cancer agent, to a subject with cancer.

As used herein, the terms “therapies” and “therapy” can refer to any protocol(s), method(s), and/or agent(s) that can be used in the prevention, treatment, management, or amelioration of cancer.

As used herein, a “protocol” includes dosing schedules and dosing regimens. The protocols herein are methods of use and include therapeutic protocols.

As used herein, a composition that “substantially” comprises a compound means that the composition contains more than about 80% by weight, more preferably more than about 90% by weight, even more preferably more than about 95% by weight, and most preferably more than about 97% by weight of the compound.

The present method utilizes compounds represented by Formulae (I) or (Ia) or in Table 1 or Table 2:

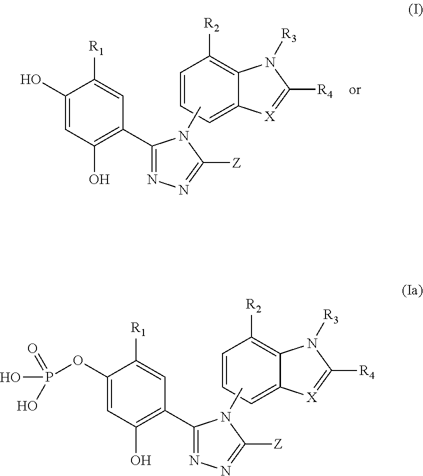

or a tautomer, or a pharmaceutically acceptable salt thereof, wherein:
Z is OH, SH, or NHR₇;
X is CR₄ or N;
R₁ is —H, —OH, —SH, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanidino, a haloalkyl, a heteroalkyl, an alkoxy or cycloalkoxy, a haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₇C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂;
R₂ is —H, —OH, —SH, —NR₇H, —OR₁₅, —SR₁₅, —NHR₁₅, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, or —NR₇C(NR₈)NR₁₀R₁₁;
R₃ is —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, a haloalkyl, a heteroalkyl, —C(O)R₇, —(CH₂)ₘC(O)OR₇, —C(O)OR₇, —OC(O)R₇, —C(O)NR₁₀R₁₁, —S(O)ₚR₇, —S(O)ₚOR₇, or —S(O)ₚNR₁₀R₁₁;
R₄ is —H, —OH, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, halo, cyano, nitro, guanidino, a haloalkyl, a heteroalkyl, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, —S(O)ₚNR₁₀R₁₁, or R₄₃ and R₄₄ taken together with the carbon atoms to which they are attached form an optionally substituted cycloalkenyl, an optionally substituted aryl, an optionally substituted heterocyclyl, or an optionally substituted heteroaryl;
R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl;
R₁₅, for each occurrence, is independently, a lower alkyl;
p, for each occurrence, is, independently, 1 or 2; and
m, for each occurrence, is independently, 1, 2, 3, or 4.

In one embodiment, in formula (I) or (Ia), X is CR₄. In another embodiment, in formula (I) or (Ia), X is N. In another embodiment, in formula (I) or (Ia), R₁ is selected from the group consisting of —H, lower alkyl, lower alkoxy, lower cycloalkyl, and lower cycloalkoxy. In another embodiment, in formula (I) or (Ia), R₁ is selected from the group consisting of —H, methyl, ethyl, propyl, isopropyl, cyclopropyl, methoxy, ethoxy, propoxy, and cyclopropoxy. In another embodiment, in formula (I) or (Ia), R₃ is selected from the group consisting of —H, a lower alkyl, a lower cycloalkyl, —C(O)N(R₂₇)₂, and —C(O)OH, wherein R₂₇ is —H or a lower alkyl. In another embodiment, in formula (I) or (Ia), R₃ is selected from the group consisting of —H, methyl, ethyl, n-propyl, isopropyl, cyclopropyl, n-butyl, sec-butyl, tert-butyl, n-pentyl, n-hexyl, —C(O)OH, —(CH₂)ₘC(O)OH, —CH₂OCH₃, —CH₂CH₂OCH₃, and —C(O)N(CH₃)₂. In one embodiment, R₄ is H or a lower alkyl. In another embodiment, in formula (I) or (Ia), R₄ is selected from the group consisting of —H, methyl, ethyl, propyl, isopropyl or cyclopropyl. In another embodiment, in formula (I) or (Ia), R₁ is selected from the group consisting of —H, —OH, —SH, —NH₂, a lower alkoxy and a lower alkyl amino. In another embodiment, in formula (I) or (Ia), R₁ is selected from the group consisting of —H, —OH, methoxy and ethoxy. In another embodiment, in formula (I) or (Ia), Z is —OH. In another embodiment, in formula (I) or (Ia), Z is —SH. In another embodiment, in formula (I) or (Ia), R₂ is selected from the group consisting of —H, —OH, —SH, —NH₂, a lower alkoxy and a lower alkyl amino. In another embodiment, in formula (I) or (Ia), R₂ is selected from the group consisting of —H, —OH, methoxy, and ethoxy. In another embodiment, in formula (I) or (Ia), R₁ is selected from the group consisting of —H, methyl, ethyl, propyl, isopropyl, cyclopropyl, methoxy, ethoxy, propoxy, and cyclopropoxy; R₃ is selected from the group consisting of —H, methyl, ethyl, n-propyl, isopropyl, cyclopropyl, n-butyl, sec-butyl, tert-butyl, n-pentyl, n-hexyl, —C(O)OH, —(CH₂)ₘC(O)OH, —CH₂OCH₃, —CH₂CH₂OCH₃, and —C(O)N(CH₃)₂; R₄ is selected from the group consisting of —H, methyl, ethyl, propyl, isopropyl or cyclopropyl; R₂ is selected from the group consisting of —H, —OH, —SH, —NH₂, a lower alkoxy and a lower alkyl amino; and Z is OH. In another embodiment, in formula (I) or (Ia), R₁ is selected from the group consisting of —H, methyl, ethyl, propyl, isopropyl, cyclopropyl, methoxy, ethoxy, propoxy, and cyclopropoxy; R₃ is selected from the group consisting of —H, methyl, ethyl, n-propyl, isopropyl, cyclopropyl, n-butyl, sec-butyl, tert-butyl, n-pentyl, n-hexyl, —C(O)OH, —(CH₂)ₘC(O)OH, —CH₂OCH₃, —CH₂CH₂OCH₃, and —C(O)N(CH₃)₂; R₄ is selected from the group consisting of —H, methyl, ethyl, propyl, isopropyl or cyclopropyl; R₂ is selected from the group consisting of —H, —OH, —SH, —NH₂, a lower alkoxy and a lower alkyl amino; and Z is SH. In another embodiment, the compound is selected from the group consisting of:

3-(2,4-dihydroxy-5-ethyl-phenyl)-4-(1,3-dimethyl-indol-5-yl)-5-hydroxy-[1,2,4]triazole, 3-(2,4-dihydroxy-5-isopropyl-phenyl)-4-(1,3-dimethyl-indol-5-yl)-5-hydroxy-[1,2,4]triazole, 3-(2,4-dihydroxy-5-isopropyl-phenyl)-4-(1-methyl-indol-5-yl)-5-hydroxy-[1,2,4]triazole, 3-(2,4-dihydroxy-5-isopropyl-phenyl)-4-(1-isopropyl-indol-4-yl)-5-hydroxy-[1,2,4]triazole, 3-(2,4-dihydroxy-5-isopropyl-phenyl)-4-(1-methyl-indazol-5-yl)-5-mercapto-[1,2,4]triazole, 3-(2,4-dihydroxy-5-isopropyl-phenyl)-4-(1-methyl-indazol-6-yl)-5-mercapto-[1,2,4]triazole, 3-(2,4-dihydroxyphenyl)-4-(1-ethyl-indol-4-yl)-5-mercapto-[1,2,4]triazole, 3-(2,4-dihydroxyphenyl)-4-(1-isopropyl-indol-4-yl)-5-mercapto-[1,2,4]triazole, 3-(2,4-dihydroxyphenyl)-4-(indol-4-yl)-5-mercapto-[1,2,4]triazole, 3-(2,4-dihydroxyphenyl)-4-(1-methoxyethyl-indol-4-yl)-5-mercapto-[1,2,4]triazole, 3-(2,4-dihydroxy-5-ethyl-phenyl)-4-(1-isopropyl-indol-4-yl)-5-mercapto-[1,2,4]triazole, 3-(2,4-dihydroxyphenyl)-4-(1-dimethylcarbamoyl-indol-4-yl)-5-mercapto-[1,2,4]triazole, 3-(2,4-dihydroxy-5-ethyl-phenyl)-4-(1-propyl-indol-4-yl)-5-mercapto-[1,2,4]triazole, 3-(2,4-dihydroxy-5-ethyl-phenyl)-4-(1,2,3-trimethyl-indol-5-yl)-5-mercapto-[1,2,4]triazole, 3-(2,4-dihydroxy-5-ethyl-phenyl)-4-(2,3-dimethyl-indol-5-yl)-5-mercapto-[1,2,4]triazole, 3-(2,4-dihydroxy-5-ethyl-phenyl)-4-(1-acetyl-2,3-dimethyl-indol-5-yl)-5-mercapto-[1,2,4]triazole, 3-(2,4-dihydroxy-5-ethyl-phenyl)-4-(1-propyl-2,3-dimethyl-indol-5-yl)-5-mercapto-[1,2,4]triazole, 3-(2,4-dihydroxy-5-ethyl-phenyl)-4-(1-n-butyl-indol-4-yl)-5-mercapto-[1,2,4]triazole, 3-(2,4-dihydroxy-5-ethyl-phenyl)-4-(1-n-pentyl-indol-4-yl)-5-mercapto-[1,2,4]triazole, 3-(2,4-dihydroxy-5-ethyl-phenyl)-4-(1-n-hexyl-indol-4-yl)-5-mercapto-[1,2,4]triazole, 3-(2,4-dihydroxy-5-cyclopropyl-phenyl)-4-(1-(1-methylcyclopropyl)-indol-4-yl)-5-mercapto-[1,2,4]triazole, 3-(2,4-dihydroxy-5-cyclopropyl-phenyl)-4-(1,2,3-trimethyl-indol-5-yl)-5-mercapto-[1,2,4]triazole, 3-(2,4-dihydroxy-5-ethyl-phenyl)-4-(1-methyl-3-ethyl-indol-5-yl)-5-mercapto-[1,2,4]triazole, 3-(2,4-dihydroxy-5-ethyl-phenyl)-4-(1,3-dimethyl-indol-5-yl)-5-mercapto-[1,2,4]triazole, 3-(2,4-dihydroxy-5-ethyl-phenyl)-4-(1-methyl-3-isopropyl-indol-5-yl)-5-mercapto-[1,2,4]triazole, 3-(2,4-dihydroxy-5-ethyl-phenyl)-4-(1,2-dimethyl-indol-5-yl)-5-mercapto-[1,2,4]triazole, 3-(2,4-dihydroxy-5-ethyl-phenyl)-4-(N-methyl-indol-5-yl)-5-mercapto-[1,2,4]triazole, 3-(2,4-dihydroxy-5-isopropyl-phenyl)-4-(1,3-dimethyl-indol-5-yl)-5-mercapto-[1,2,4]triazole, 3-(2,4-dihydroxy-5-cyclopropyl-phenyl)-4-(1,3-dimethyl-indol-5-yl)-5-mercapto-[1,2,4]triazole, 3-(2,4-dihydroxy-5-cyclopropyl-phenyl)-4-(1-methyl-indol-5-yl)-5-mercapto-[1,2,4]triazole, 3-(2,4-dihydroxy-5-isopropyl-phenyl)-4-(1H-indol-5-yl)-5-mercapto-[1,2,4]triazole, 3-(2,4-dihydroxy-5-ethyl-phenyl)-4-(1,2-dimethyl-indol-5-yl)-5-mercapto-[1,2,4]triazole, 3-(2,4-dihydroxy-5-isopropyl-phenyl)-4-(1-ethyl-indol-5-yl)-5-mercapto-[1,2,4]triazole, 3-(2,4-dihydroxy-5-isopropyl-phenyl)-4-(1-propyl-indol-5-yl)-5-mercapto-[1,2,4]triazole, or a tautomer, or a pharmaceutically acceptable salt thereof.

In another embodiment, in formula (I) or (Ia), X is N.

In another embodiment, the compound is selected from the group consisting of 3-(2,4-dihydroxy-5-ethyl-phenyl)-4-(1-ethyl-benzimidazol-4-yl)-5-mercapto-[1,2,4]triazole, 3-(2,4-dihydroxy-5-ethyl-phenyl)-4-(1-ethyl-benzimidazol-4-yl)-5-mercapto-[1,2,4]triazole HCL salt, 3-(2,4-dihydroxy-5-ethyl-phenyl)-4-(2-methyl-3-ethyl-benzimidazol-5-yl)-5-mercapto-[1,2,4]triazole, 3-(2,4-dihydroxy-5-ethyl-phenyl)-4-(1-ethyl-2-methyl-benzimidazol-5-yl)-5-mercapto-[1,2,4]triazole, 3-(2,4-dihydroxy-5-isopropyl-phenyl)-4-(1-methyl-2-trifluoromethyl-benzimidazol-5-yl)-5-mercapto-[1,2,4]triazole, or a tautomer, or a pharmaceutically acceptable salt thereof.

i) Exemplary Compounds

Exemplary compounds described herein are depicted in Table 1 below, including tautomers or pharmaceutically acceptable salts.

TABLE 1

| | Structure | Tautomeric Structure | Name |
|---|---|---|---|
| 1 | 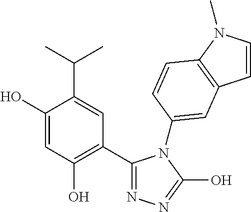 [SMILES: CC(C)c1cc(-c2nnc(O)n2-c2ccc3c(ccn3C)c2)c(O)cc1O] | 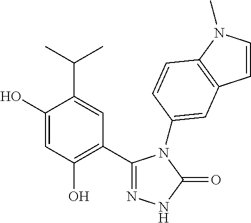 [SMILES: CC(C)c1cc(-c2n[nH]c(=O)n2-c2ccc3c(ccn3C)c2)c(O)cc1O] | 3-(2,4-dihydroxy-5- isopropyl-phenyl)-4- (1-methyl-indol-5-yl)- 5-hydroxy-[1,2,4] triazole |
| 2 | 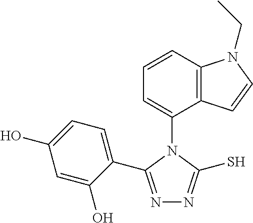 [SMILES: CCn1ccc2c(-n3c(S)nnc3-c3ccc(O)cc3O)cccc21] | 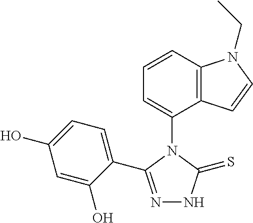 [SMILES: CCn1ccc2c(-n3c(-c4ccc(O)cc4O)n[nH]c3=S)cccc21] | 3-(2,4- Dihydroxyphenyl)-4- (1-ethyl-indol-4-yl)-5- mercapto-[1,2,4] triazole |
| 3 | 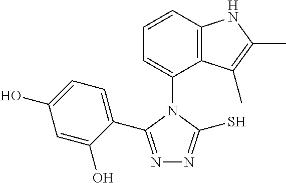 [SMILES: Cc1[nH]c2cccc(-n3c(S)nnc3-c3ccc(O)cc3O)c2c1C] | 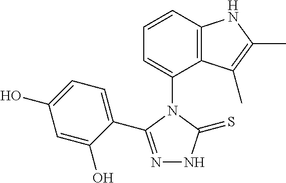 [SMILES: Cc1[nH]c2cccc(-n3c(-c4ccc(O)cc4O)n[nH]c3=S)c2c1C] | 3-(2,4-Dihydroxy- phenyl)-4-(2,3- dimethyl-1H-indol-4- yl)-5-mercapto-[1,2,4] triazole |
| 4 | 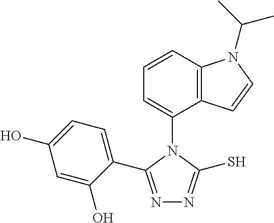 [SMILES: CC(C)n1ccc2c(-n3c(S)nnc3-c3ccc(O)cc3O)cccc21] | 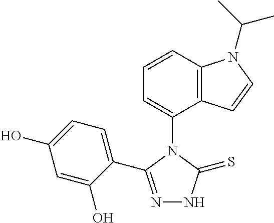 [SMILES: CC(C)n1ccc2c(-n3c(-c4ccc(O)cc4O)n[nH]c3=S)cccc21] | 3-(2,4- Dihydroxyphenyl)-4- (1-isopropyl-indol-4- yl)-5-mercapto-[1,2,4] triazole |
| 5 | 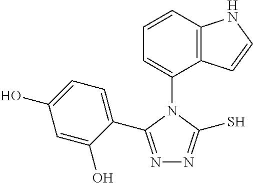 [SMILES: Oc1ccc(-c2nnc(S)n2-c2cccc3[nH]ccc23)c(O)c1] | 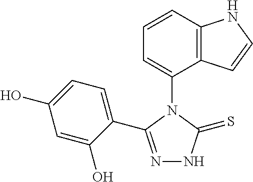 [SMILES: Oc1ccc(-c2n[nH]c(=S)n2-c2cccc3[nH]ccc23)c(O)c1] | 3-(2,4-Dihydroxy- phenyl)-4-(indol-4- yl)-5-mercapto-[1,2,4] triazole |
| 6 | 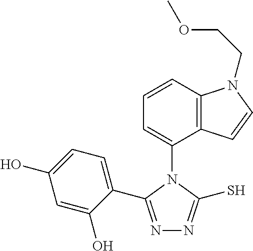 [SMILES: COCCn1ccc2c(-n3c(S)nnc3-c3ccc(O)cc3O)cccc21] | 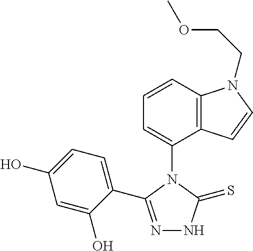 [SMILES: COCCn1ccc2c(-n3c(-c4ccc(O)cc4O)n[nH]c3=S)cccc21] | 3-(2,4-Dihydroxy- phenyl)-4-[1-(2- methoxyethoxy)- indol-4-yl)-5- mercapto-[1,2,4] triazole |
| 7 | 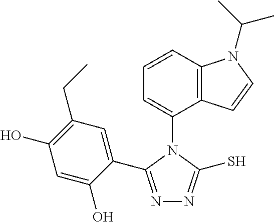 [SMILES: CCc1cc(-c2nnc(S)n2-c2cccc3c2ccn3C(C)C)c(O)cc1O] | 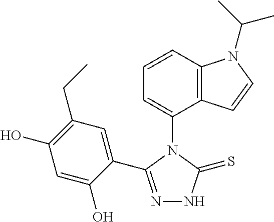 [SMILES: CCc1cc(-c2n[nH]c(=S)n2-c2cccc3c2ccn3C(C)C)c(O)cc1O] | 3-(2,4-Dihydroxy-5- ethyl-phenyl)-4-(1- isopropyl-indol-4-yl)- 5-mercapto-[1,2,4] triazole |
| 8 | 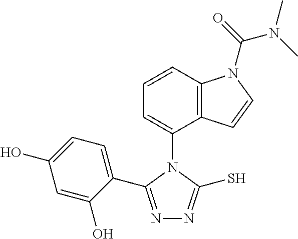 [SMILES: CN(C)C(=O)n1ccc2c(-n3c(S)nnc3-c3ccc(O)cc3O)cccc21] | 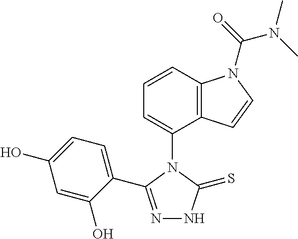 [SMILES: CN(C)C(=O)n1ccc2c(-n3c(-c4ccc(O)cc4O)n[nH]c3=S)cccc21] | 3-(2,4-Dihydroxy-5- ethyl-phenyl)-4-[1- (dimethyl- carbamoyl)-indol-4- yl]-5-mercapto-[1,2,4] triazole |
| 9 | 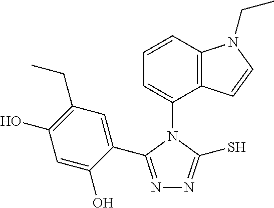 [SMILES: CCc1cc(-c2nnc(S)n2-c2cccc3c2ccn3CC)c(O)cc1O] | 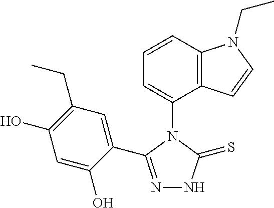 [SMILES: CCc1cc(-c2n[nH]c(=S)n2-c2cccc3c2ccn3CC)c(O)cc1O] | 3-(2,4-Dihydroxy-5- ethyl-phenyl)-4-(1- ethyl-benzoimidazol- 4-yl)-5-mercapto- [1,2,4]triazole |
| 10 | 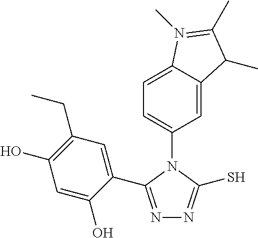 | 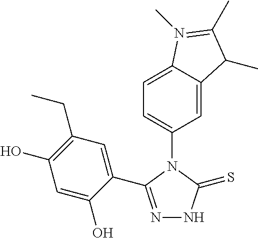 | 3-(2,4-Dihydroxy-5- ethyl-phenyl)-4- (1,2,3-trimethyl-indol- 5-yl)-5-mercapto- [1,2,4]triazole |
| 11 | 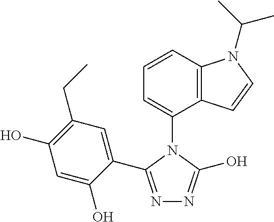 [SMILES: CCc1cc(-c2nnc(O)n2-c2cccc3c2ccn3C(C)C)c(O)cc1O] | 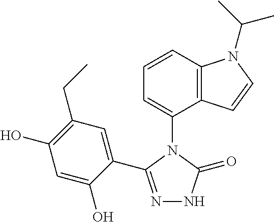 [SMILES: CCc1cc(-c2n[nH]c(=O)n2-c2cccc3c2ccn3C(C)C)c(O)cc1O] | 3-(2,4-Dihydroxy-5- ethyl-phenyl)-4-(1- isopropyl-indol-3-yl)- 5-hydroxy-[1,2,4] triazole |
| 12 | 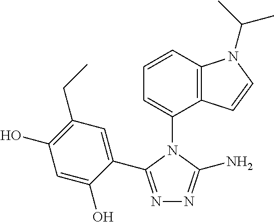 [SMILES: CCc1cc(-c2nnc(N)n2-c2cccc3c2ccn3C(C)C)c(O)cc1O] | 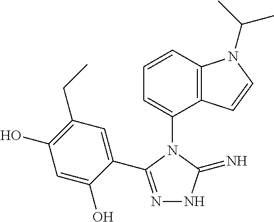 [SMILES: CCc1cc(-c2n[nH]c(=N)n2-c2cccc3c2ccn3C(C)C)c(O)cc1O] | 3-(2,4-Dihydroxy-5- ethyl-phenyl)-4-(1- isopropyl-indol-4-yl)- 5-amino-[1,2,4] triazole |
| 15 | 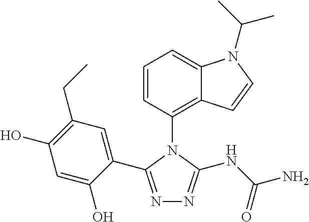 [SMILES: CCc1cc(-c2nnc(NC(N)=O)n2-c2cccc3c2ccn3C(C)C)c(O)cc1O] | | 3-(2,4-Dihydroxy-5- ethyl-phenyl)-4-(1- isopropyl-indol-4-yl)- 5-ureido-[1,2,4] triazole |
| 16 | 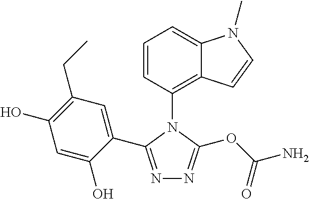 [SMILES: CCc1cc(-c2nnc(OC(N)=O)n2-c2cccc3c2ccn3C)c(O)cc1O] | | 3-(2,4-Dihydroxy-5- ethyl-phenyl)-4-(1- methyl-indol-4-yl)-5- carbamoyloxy-[1,2,4] triazole |
| 17 | 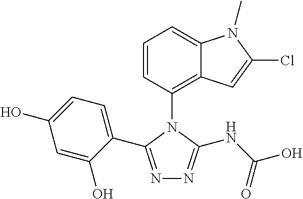 [SMILES: Cn1c(Cl)cc2c(-n3c(NC(=O)O)nnc3-c3ccc(O)cc3O)cccc21] | | 3-(2,4-Dihydroxy- phenyl)-4-(1-methyl- 2-chloro-indol-4-yl)- 5-carbamoyloxy- [1,2,4]triazole |
| 18 | 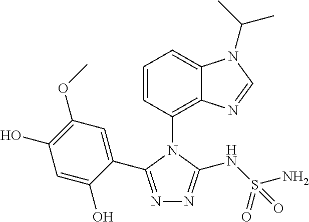 [SMILES: COc1cc(-c2nnc(NS(N)(=O)=O)n2-c2cccc3c2ncn3C(C)C)c(O)cc1O] | | 3-(2,4-Dihydroxy-5- methoxy-phenyl)-4- (1-isopropyl- benzoimidazol-4-yl)- 5-(sulfamoylamino)- [1,2,4]triazole |
| 20 | 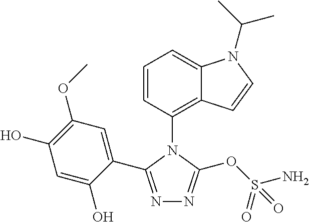 [SMILES: COc1cc(-c2nnc(OS(N)(=O)=O)n2-c2cccc3c2ccn3C(C)C)c(O)cc1O] | | 3-(2,4-Dihydroxy-5- methoxy-phenyl)-4- (1-isopropyl- benzoimidazol-4-yl)- 5-(sulfamoyloxy)- [1,2,4]triazole |
| 21 | 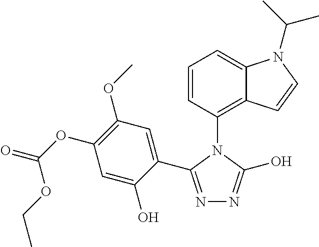 [SMILES: CCOC(=O)Oc1cc(O)c(-c2nnc(O)n2-c2cccc3c2ccn3C(C)C)cc1OC] | 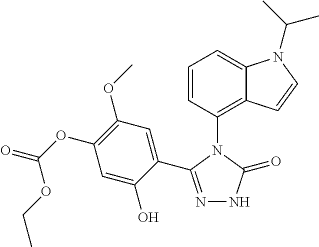 [SMILES: CCOC(=O)Oc1cc(O)c(-c2n[nH]c(=O)n2-c2cccc3c2ccn3C(C)C)cc1OC] | 3-(2-Hydroxy-4- ethoxycarbonyoxy-5- methoxy-phenyl)-4- (1-isopropyl- benzoimidazol-4-yl)- 5-hydroxy-[1,2,4] triazole |
| 22 | 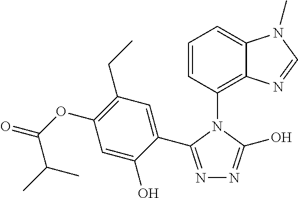 [SMILES: CCc1cc(-c2nnc(O)n2-c2cccc3c2ncn3C)c(O)cc1OC(=O)C(C)C] | 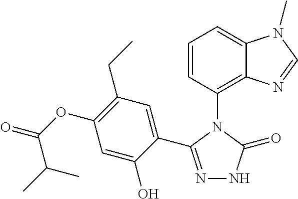 [SMILES: CCc1cc(-c2n[nH]c(=O)n2-c2cccc3c2ncn3C)c(O)cc1OC(=O)C(C)C] | 3-[2-Hydroxy-4- isobutyryloxy-5-ethyl- phenyl)-4-(1-methyl- benzo-imidazol-4-yl)- 5-hydroxy-[1,2,4] triazole |
| 23 | 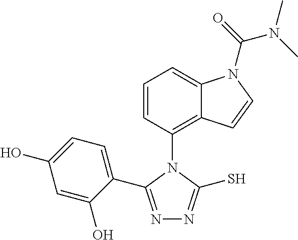 [SMILES: CN(C)C(=O)n1ccc2c(-n3c(S)nnc3-c3ccc(O)cc3O)cccc21] | 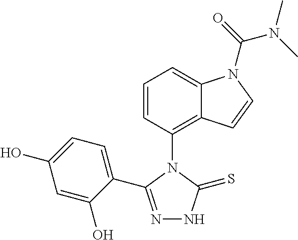 [SMILES: CN(C)C(=O)n1ccc2c(-n3c(-c4ccc(O)cc4O)n[nH]c3=S)cccc21] | 3-(2,4-Dihydroxy- phenyl)-4-(1- dimethylcarbamoyl- indol-4-yl)-5- mercapto-[1,2,4] triazole |
| 24 | 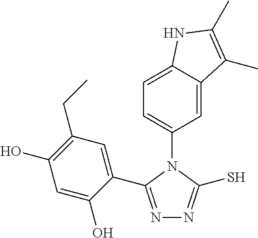 [SMILES: CCc1cc(-c2nnc(S)n2-c2ccc3[nH]c(C)c(C)c3c2)c(O)cc1O] | 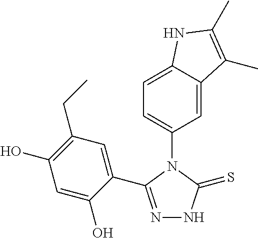 [SMILES: CCc1cc(-c2n[nH]c(=S)n2-c2ccc3[nH]c(C)c(C)c3c2)c(O)cc1O] | 3-(2,4-Dihydroxy-5- ethyl-phenyl)-4-(2,3- dimethyl-indol-5-yl)- 5-mercapto-[1,2,4] triazole |
| 25 | 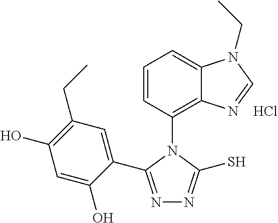 [SMILES: CCc1cc(-c2nnc(S)n2-c2cccc3c2ncn3CC)c(O)cc1O ; Cl] | 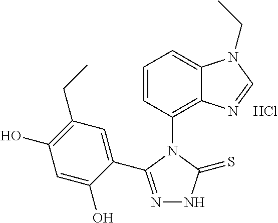 [SMILES: CCc1cc(-c2n[nH]c(=S)n2-c2cccc3c2ncn3CC)c(O)cc1O ; Cl] | 3-(2,4-Dihydroxy-5- ethyl-phenyl)-4-(1- ethyl-1H- benzoimidazol-4-yl)- 5-mercapto-[1,2,4] triazole, HCl salt |
| 26 | 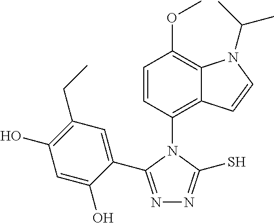 [SMILES: CCc1cc(-c2nnc(S)n2-c2ccc(OC)c3c2ccn3C(C)C)c(O)cc1O] | 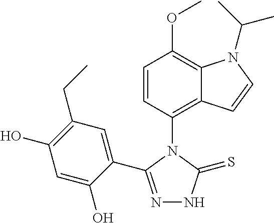 [SMILES: CCc1cc(-c2n[nH]c(=S)n2-c2ccc(OC)c3c2ccn3C(C)C)c(O)cc1O] | 3-(2,4-Dihydroxy-5- ethyl-phenyl)-4-(1- isopropyl-7-methoxy- indol-4-yl)-5- mercapto-[1,2,4] triazole |
| 27 | 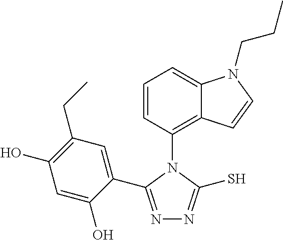 [SMILES: CCCn1ccc2c(-n3c(S)nnc3-c3cc(CC)c(O)cc3O)cccc21] | 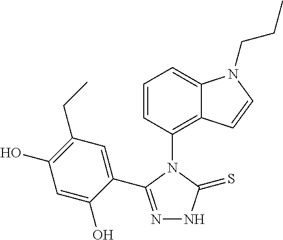 [SMILES: CCCn1ccc2c(-n3c(-c4cc(CC)c(O)cc4O)n[nH]c3=S)cccc21] | 3-(2,4-dihydroxy-5- ethyl-phenyl)-4-(1- propyl-indol-4-yl)-5- mercapto-[1,2,4] triazole |
| 28 | 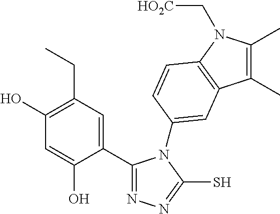 [SMILES: CCc1cc(-c2nnc(S)n2-c2ccc3c(c2)c(C)c(C)n3CC(=O)O)c(O)cc1O] | 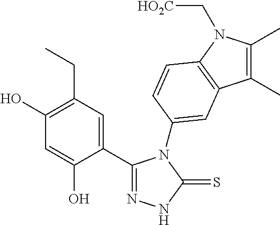 [SMILES: CCc1cc(-c2n[nH]c(=S)n2-c2ccc3c(c2)c(C)c(C)n3CC(=O)O)c(O)cc1O] | 3-(2,4-dihydroxy-5- ethyl-phenyl)-4-(1- acetyl-2,3-dimethyl- indol-5-yl)-5- mercapto-[1,2,4] triazole |
| 29 | 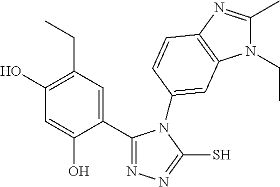 [SMILES: CCc1cc(-c2nnc(S)n2-c2ccc3nc(C)n(CC)c3c2)c(O)cc1O] | 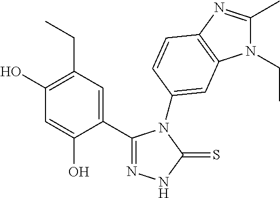 [SMILES: CCc1cc(-c2n[nH]c(=S)n2-c2ccc3nc(C)n(CC)c3c2)c(O)cc1O] | 3-(2,4-dihydroxy-5- ethyl-phenyl)-4-(2- methyl-3-ethyl- benzimidazol-5-yl)-5- mercapto-[1,2,4] triazole |
| 30 | 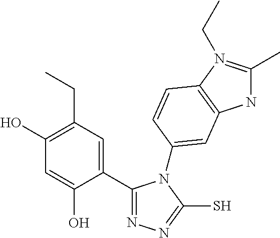 | 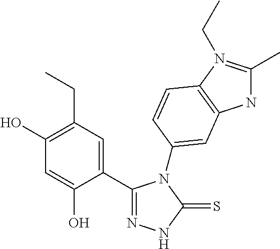 | 3-(2,4-dihydroxy-5- ethyl-phenyl)-4-(1- ethyl-2-methyl- benzimidazol-5-yl)-5- mercapto-[1,2,4] triazole |
| 31 | 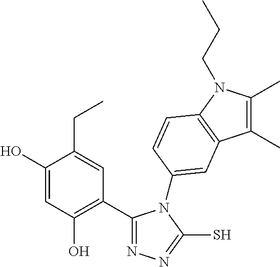 [SMILES: CCCn1c(C)c(C)c2cc(-n3c(S)nnc3-c3cc(CC)c(O)cc3O)ccc21] | 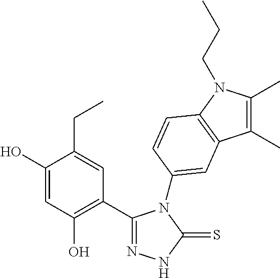 [SMILES: CCCn1c(C)c(C)c2cc(-n3c(-c4cc(CC)c(O)cc4O)n[nH]c3=S)ccc21] | 3-(2,4-dihydroxy-5- ethyl-phenyl)-4-(1- propyl-2,3-dimethyl- indol-5-yl)-5- mercapto-[1,2,4] triazole |
| 34 | 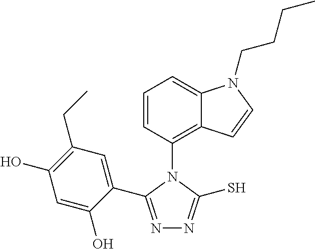 [SMILES: CCCCn1ccc2c(-n3c(S)nnc3-c3cc(CC)c(O)cc3O)cccc21] | 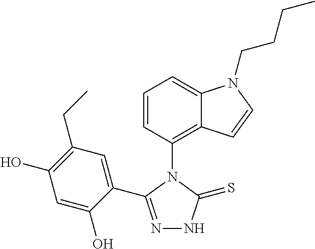 [SMILES: CCCCn1ccc2c(-n3c(-c4cc(CC)c(O)cc4O)n[nH]c3=S)cccc21] | 3-(2,4-dihydroxy-5- ethyl-phenyl)-4-(1-n- butyl-indol-4-yl)-5- mercapto-[1,2,4] triazole |
| 35 | 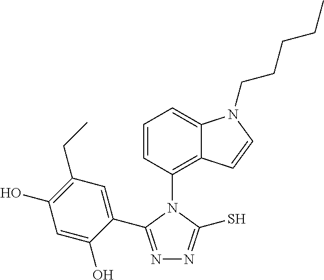 [SMILES: CCCCCn1ccc2c(-n3c(S)nnc3-c3cc(CC)c(O)cc3O)cccc21] | 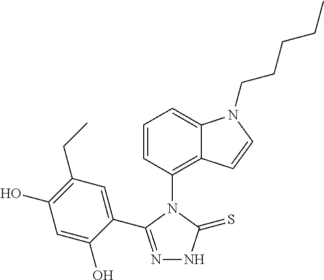 [SMILES: CCCCCn1ccc2c(-n3c(-c4cc(CC)c(O)cc4O)n[nH]c3=S)cccc21] | 3-(2,4-dihydroxy-5- ethyl-phenyl)-4-(1-n- pentyl-indol-4-yl)-5- mercapto-[1,2,4] triazole |
| 36 | 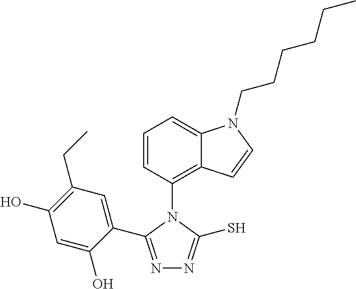 [SMILES: CCCCCCn1ccc2c(-n3c(S)nnc3-c3cc(CC)c(O)cc3O)cccc21] | 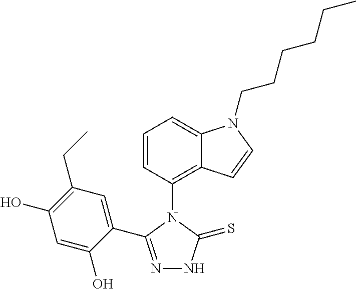 [SMILES: CCCCCCn1ccc2c(-n3c(-c4cc(CC)c(O)cc4O)n[nH]c3=S)cccc21] | 3-(2,4-dihydroxy-5- ethyl-phenyl)-4-(1-n- hexyl-indol-4-yl)-5- mercapto-[1,2,4] triazole |
| 37 | 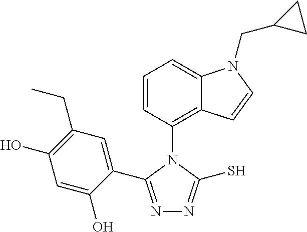 [SMILES: CCc1cc(-c2nnc(S)n2-c2cccc3c2ccn3CC2CC2)c(O)cc1O] | 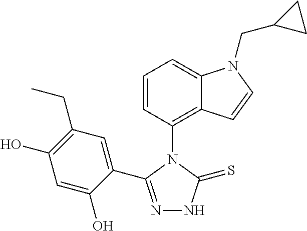 [SMILES: CCc1cc(-c2n[nH]c(=S)n2-c2cccc3c2ccn3CC2CC2)c(O)cc1O] | 3-(2,4-dihydroxy-5- cyclopropyl-phenyl)- 4-(1-(1- methylcyclopropyl)- indol-4-yl)-5- mercapto-[1,2,4] triazole |
| 38 | 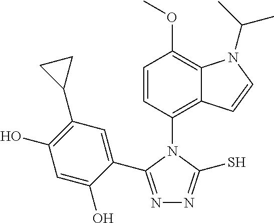 [SMILES: COc1ccc(-n2c(S)nnc2-c2cc(C3CC3)c(O)cc2O)c2ccn(C(C)C)c12] | 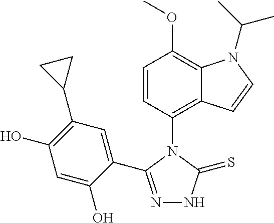 [SMILES: COc1ccc(-n2c(-c3cc(C4CC4)c(O)cc3O)n[nH]c2=S)c2ccn(C(C)C)c12] | 3-(2,4-dihydroxy-5- cyclopropyl-phenyl)- 4-(1-isopropyl-7- methoxy-indol-4-yl)- 5-mercapto-[1,2,4] triazole |
| 39 | 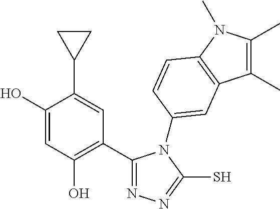 [SMILES: Cc1c(C)n(C)c2ccc(-n3c(S)nnc3-c3cc(C4CC4)c(O)cc3O)cc12] | 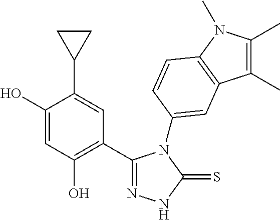 [SMILES: Cc1c(C)n(C)c2ccc(-n3c(-c4cc(C5CC5)c(O)cc4O)n[nH]c3=S)cc12] | 3-(2,4-dihydroxy-5- cyclopropyl-phenyl)- 4-(1,2,3-trimethyl- indol-5-yl)-5- mercapto-[1,2,4] triazole |
| 40 | 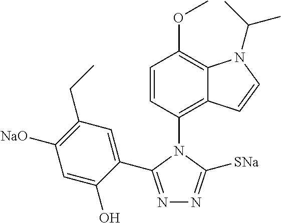 | | 3-(2,4-dihydroxy-5- ethyl-phenyl)-4-(1- isopropyl-7-methoxy- indol-4-yl)-5- mercapto-[1,2,4] triazole disodium salt |
| 41 | 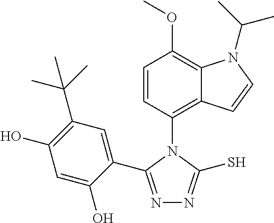 [SMILES: COc1ccc(-n2c(S)nnc2-c2cc(C(C)(C)C)c(O)cc2O)c2ccn(C(C)C)c12] | 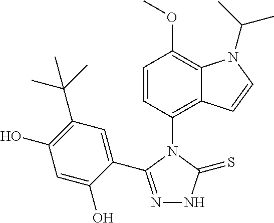 [SMILES: COc1ccc(-n2c(-c3cc(C(C)(C)C)c(O)cc3O)n[nH]c2=S)c2ccn(C(C)C)c12] | 3-(2,4-dihydroxy-5- tert-butyl-phenyl)-4- (1-isopropyl-7- methoxy-indol-4-yl)- 5-mercapto-[1,2,4] triazole |
| 42 | 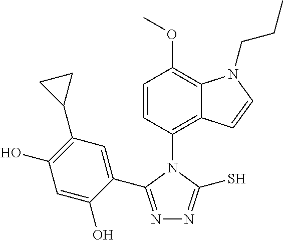 [SMILES: CCCn1ccc2c(-n3c(S)nnc3-c3cc(C4CC4)c(O)cc3O)ccc(OC)c21] | 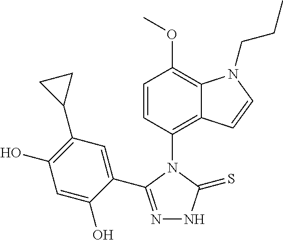 [SMILES: CCCn1ccc2c(-n3c(-c4cc(C5CC5)c(O)cc4O)n[nH]c3=S)ccc(OC)c21] | 3-(2,4-dihydroxy-5- cyclopropyl-phenyl)- 4-(1-propyl-7- methoxy-indol-4-yl)- 5-mercapto-[1,2,4] triazole |
| 43 | 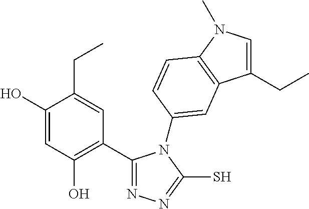 [SMILES: CCc1cc(-c2nnc(S)n2-c2ccc3c(c2)c(CC)cn3C)c(O)cc1O] | 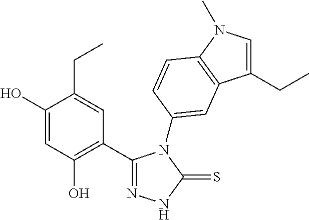 | 3-(2,4-dihydroxy-5- ethyl-phenyl)-4-(1- methyl-3-ethyl-indol- 5-yl)-5-mercapto- [1,2,4]triazole |
| 44 | 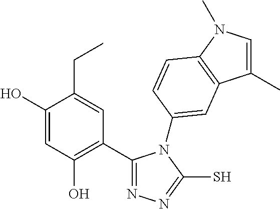 [SMILES: CCc1cc(-c2nnc(S)n2-c2ccc3c(c2)c(C)cn3C)c(O)cc1O] | 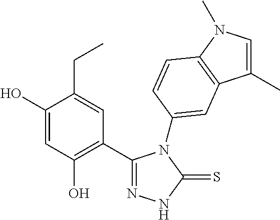 [SMILES: CCc1cc(-c2n[nH]c(=S)n2-c2ccc3c(c2)c(C)cn3C)c(O)cc1O] | 3-(2,4-dihydroxy-5- ethyl-phenyl)-4-(1,3- dimethyl-indol-5-yl)- 5-mercapto-[1,2,4] triazole |
| 45 | 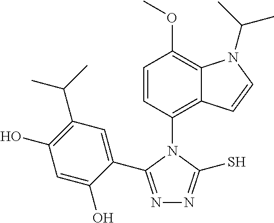 [SMILES: COc1ccc(-n2c(S)nnc2-c2cc(C(C)C)c(O)cc2O)c2ccn(C(C)C)c12] | 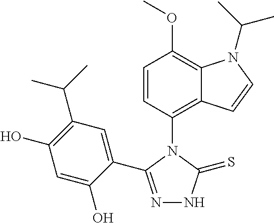 [SMILES: COc1ccc(-n2c(-c3cc(C(C)C)c(O)cc3O)n[nH]c2=S)c2ccn(C(C)C)c12] | 3-(2,4-dihydroxy-5- isopropyl-phenyl)-4- (1-isopropyl-7- methoxy-indol-4-yl)- 5-mercapto-[1,2,4] triazole |
| 46 | 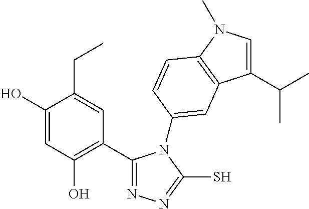 [SMILES: CCc1cc(-c2nnc(S)n2-c2ccc3c(c2)c(C(C)C)cn3C)c(O)cc1O] | 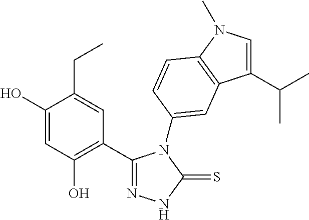 [SMILES: CCc1cc(-c2n[nH]c(=S)n2-c2ccc3c(c2)c(C(C)C)cn3C)c(O)cc1O] | 3-(2,4-dihydroxy-5- ethyl-phenyl)-4-(1- methyl-3-isopropyl- indol-5-yl)-5- mercapto-[1,2,4] triazole |
| 48 | 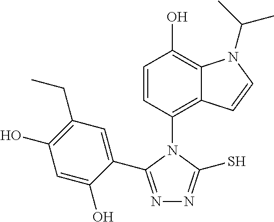 [SMILES: CCc1cc(-c2nnc(S)n2-c2ccc(O)c3c2ccn3C(C)C)c(O)cc1O] | 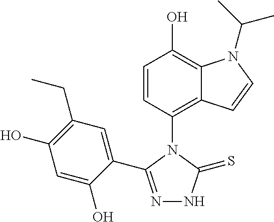 [SMILES: CCc1cc(-c2n[nH]c(=S)n2-c2ccc(O)c3c2ccn3C(C)C)c(O)cc1O] | 3-(2,4-dihydroxy-5- ethyl-phenyl)-4-(1- isopropyl-7-hydroxy- indol-4-yl)-5- mercapto-[1,2,4] triazole |
| 49 | 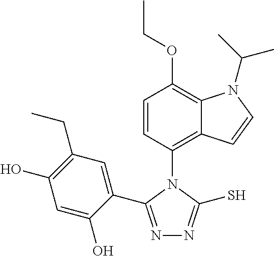 [SMILES: CCOc1ccc(-n2c(S)nnc2-c2cc(CC)c(O)cc2O)c2ccn(C(C)C)c12] | 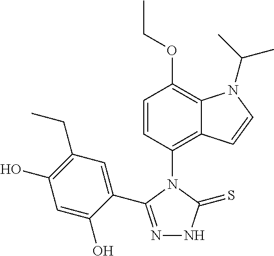 [SMILES: CCOc1ccc(-n2c(-c3cc(CC)c(O)cc3O)n[nH]c2=S)c2ccn(C(C)C)c12] | 3-(2,4-dihydroxy-5- ethyl-phenyl)-4-(1- isopropyl-7-ethoxy- indol-4-yl)-5- mercapto-[1,2,4] triazole |
| 50 | 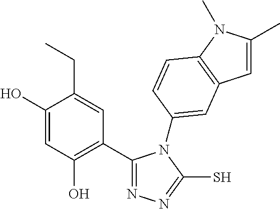 [SMILES: CCc1cc(-c2nnc(S)n2-c2ccc3c(c2)cc(C)n3C)c(O)cc1O] | 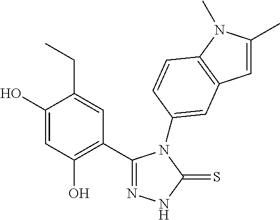 [SMILES: CCc1cc(-c2n[nH]c(=S)n2-c2ccc3c(c2)cc(C)n3C)c(O)cc1O] | 3-(2,4-dihydroxy-5- ethyl-phenyl)-4-(1,2- dimethyl-indol-5-yl)- 5-mercapto-[1,2,4] triazole |
| 51 | 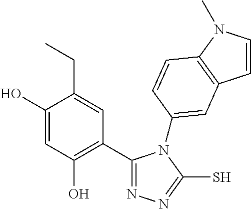 [SMILES: CCc1cc(-c2nnc(S)n2-c2ccc3c(ccn3C)c2)c(O)cc1O] | 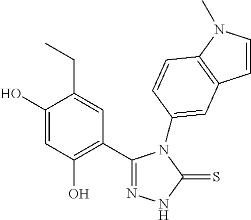 [SMILES: CCc1cc(-c2n[nH]c(=S)n2-c2ccc3c(ccn3C)c2)c(O)cc1O] | 3-(2,4-dihydroxy-5- ethyl-phenyl)-4-(N- methyl-indol-5-yl)-5- mercapto-[1,2,4] triazole |
| 55 | 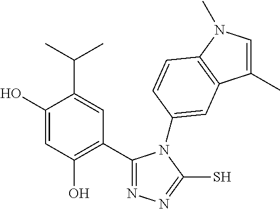 [SMILES: Cc1cn(C)c2ccc(-n3c(S)nnc3-c3cc(C(C)C)c(O)cc3O)cc12] | 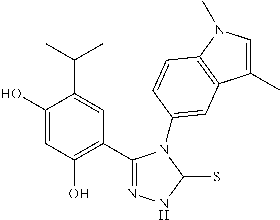 [SMILES: Cc1cn(C)c2ccc(N3C(c4cc(C(C)C)c(O)cc4O)=NNC3S)cc12] | 3-(2,4-dihydroxy-5- isopropyl-phenyl)-4- (1,3-dimethyl-indol-5- yl)-5-mercapto-[1,2,4] triazole |
| 56 | 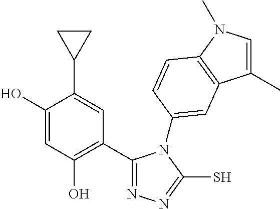 [SMILES: Cc1cn(C)c2ccc(-n3c(S)nnc3-c3cc(C4CC4)c(O)cc3O)cc12] | 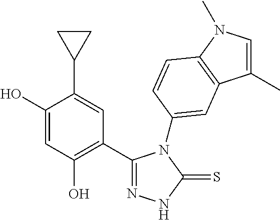 [SMILES: Cc1cn(C)c2ccc(-n3c(-c4cc(C5CC5)c(O)cc4O)n[nH]c3=S)cc12] | 3-(2,4-dihydroxy-5- cyclopropyl-phenyl)- 4-(1,3-dimethyl-indol- 5-yl)-5-mercapto- [1,2,4]triazole |
| 57 | 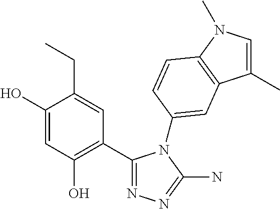 [SMILES: CCc1cc(-c2nnc(N)n2-c2ccc3c(c2)c(C)cn3C)c(O)cc1O] | 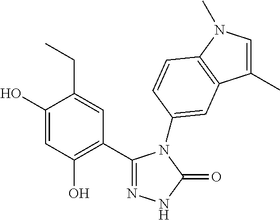 [SMILES: CCc1cc(-c2n[nH]c(=O)n2-c2ccc3c(c2)c(C)cn3C)c(O)cc1O] | 3-(2,4-dihydroxy-5- ethyl-phenyl)-4-(1,3- dimethyl-indol-5-yl)- 5-hydroxy-[1,2,4] triazole |
| 58 | 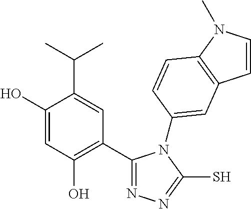 [SMILES: CC(C)c1cc(-c2nnc(S)n2-c2ccc3c(ccn3C)c2)c(O)cc1O] | 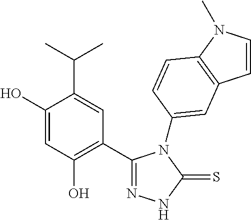 [SMILES: CC(C)c1cc(-c2n[nH]c(=S)n2-c2ccc3c(ccn3C)c2)c(O)cc1O] | 3-(2,4-dihydroxy-5- isopropyl-phenyl)-4- (N-methyl-indol-5- yl)-5-mercapto-[1,2,4] triazole |
| 59 | 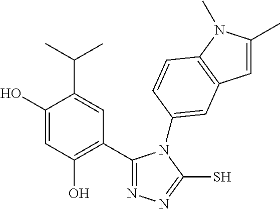 [SMILES: Cc1cc2cc(-n3c(S)nnc3-c3cc(C(C)C)c(O)cc3O)ccc2n1C] | 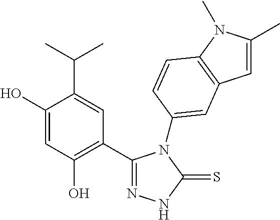 [SMILES: Cc1cc2cc(-n3c(-c4cc(C(C)C)c(O)cc4O)n[nH]c3=S)ccc2n1C] | 3-(2,4-dihydroxy-5- isopropyl-phenyl)-4- (1,2-dimethyl-indol-5- yl)-5-mercapto-[1,2,4] triazole |
| 60 | 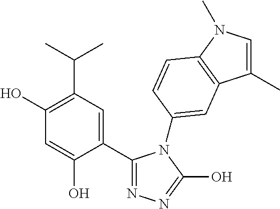 [SMILES: Cc1cn(C)c2ccc(-n3c(O)nnc3-c3cc(C(C)C)c(O)cc3O)cc12] | 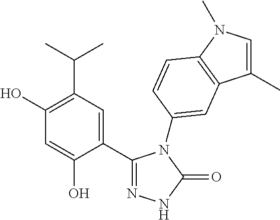 [SMILES: Cc1cn(C)c2ccc(-n3c(-c4cc(C(C)C)c(O)cc4O)n[nH]c3=O)cc12] | 3-(2,4-dihydroxy-5- isopropyl-phenyl)-4- (1,3-dimethyl-indol-5- yl)-5-hydroxy-[1,2,4] triazole |
| 62 | 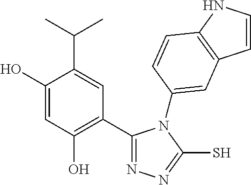 [SMILES: CC(C)c1cc(-c2nnc(S)n2-c2ccc3[nH]ccc3c2)c(O)cc1O] | 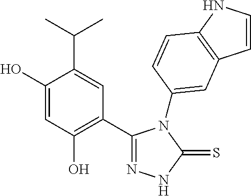 [SMILES: CC(C)c1cc(-c2n[nH]c(=S)n2-c2ccc3[nH]ccc3c2)c(O)cc1O] | 3-(2,4-dihydroxy-5- isopropyl-phenyl)-4- (1H-indol-5-yl)-5- mercapto-[1,2,4] triazole |
| 63 | 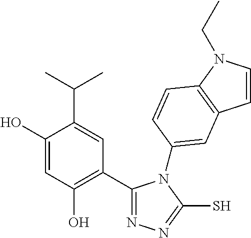 [SMILES: CCn1ccc2cc(-n3c(S)nnc3-c3cc(C(C)C)c(O)cc3O)ccc21] | 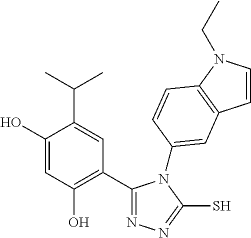 [SMILES: CCn1ccc2cc(-n3c(S)nnc3-c3cc(C(C)C)c(O)cc3O)ccc21] | 3-(2,4-dihydroxy-5- isopropyl-phenyl)-4- (1-ethyl-indol-5-yl)-5- mercapto-[1,2,4] triazole |
| 64 | 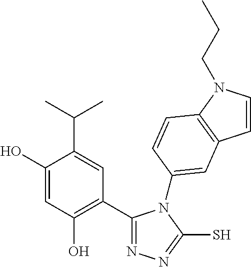 [SMILES: CCCn1ccc2cc(-n3c(S)nnc3-c3cc(C(C)C)c(O)cc3O)ccc21] | 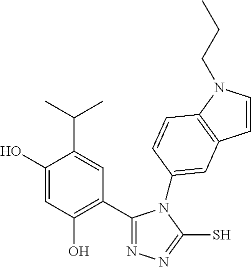 [SMILES: CCCn1ccc2cc(-n3c(S)nnc3-c3cc(C(C)C)c(O)cc3O)ccc21] | 3-(2,4-dihydroxy-5- isopropyl-phenyl)-4- (1-propyl-indol-5-yl)- 5-mercapto-[1,2,4] triazole |
| 65 | 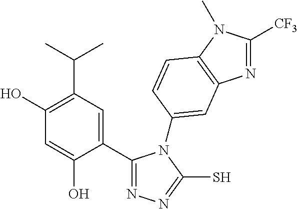 [SMILES: CC(C)c1cc(-c2nnc(S)n2-c2ccc3c(c2)nc(C(F)(F)F)n3C)c(O)cc1O] | 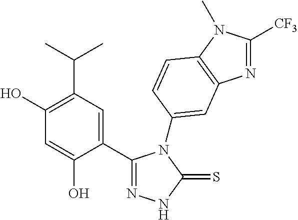 [SMILES: CC(C)c1cc(-c2n[nH]c(=S)n2-c2ccc3c(c2)nc(C(F)(F)F)n3C)c(O)cc1O] | 3-(2,4-dihydroxy-5- isopropyl-phenyl)-4- (1-methyl-2- trifluoromethyl- benzimidazol-5-yl)-5- mercapto-[1,2,4] triazole |
| 66 | 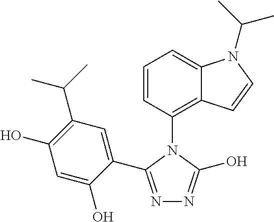 [SMILES: CC(C)c1cc(-c2nnc(O)n2-c2cccc3c2ccn3C(C)C)c(O)cc1O] | 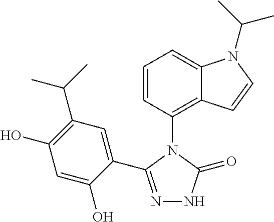 [SMILES: CC(C)c1cc(-c2n[nH]c(=O)n2-c2cccc3c2ccn3C(C)C)c(O)cc1O] | 3-(2,4-dihydroxy-5- isopropyl-phenyl)-4- (1-isopropyl-indol-4- yl)-5-hydroxy-[1,2,4] triazole |

TABLE 2
Compounds according to Formula (Ia)

| No. | Structure | Tautomeric structure | Name |
|---|---|---|---|
| 1a | 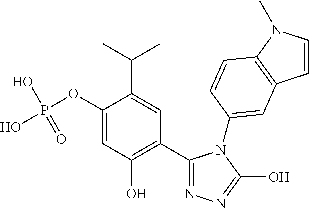 [SMILES: CC(C)c1cc(-c2nnc(O)n2-c2ccc3c(ccn3C)c2)c(O)cc1OP(=O)(O)O] | 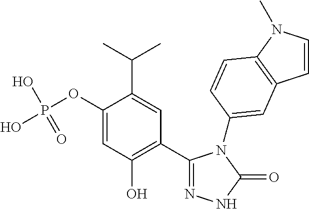 [SMILES: CC(C)c1cc(-c2n[nH]c(=O)n2-c2ccc3c(ccn3C)c2)c(O)cc1OP(=O)(O)O] | 5-hydroxy-4-(5- hydroxy-4-(1- methyl-1H- indol-5-yl)-4H- 1,2,4-triazol-3- yl)-2- isopropylphenyl dihydrogen phosphate |
| 2a | 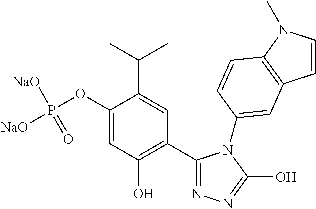 | 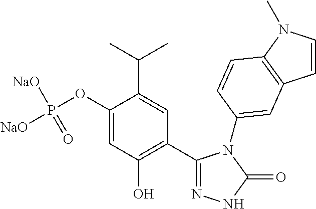 | sodium 5- hydroxy-4-(5- hydroxy-4-(1- methyl-1H- indol-5-yl)-4H- 1,2,4-triazol-3- yl)-2- isopropylphenyl phosphate |
| 3a | 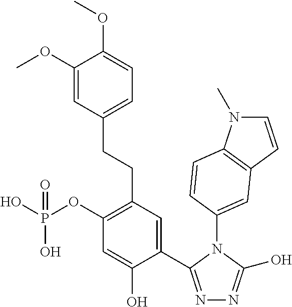 [SMILES: COc1ccc(CCc2cc(-c3nnc(O)n3-c3ccc4c(ccn4C)c3)c(O)cc2OP(=O)(O)O)cc1OC] | 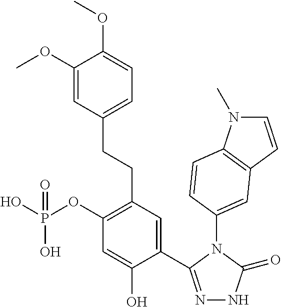 [SMILES: COc1ccc(CCc2cc(-c3n[nH]c(=O)n3-c3ccc4c(ccn4C)c3)c(O)cc2OP(=O)(O)O)cc1OC] | 2-(3,4- dimethoxy- phenethyl)-5- hydroxy- 4-(5-hydroxy-4- (1-methyl-1H- indol-5-yl)-4H- 1,2,4-triazol-3- yl)phenyl dihydrogen phosphate |
| 4a | 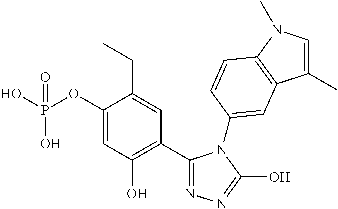 [SMILES: CCc1cc(-c2nnc(O)n2-c2ccc3c(c2)c(C)cn3C)c(O)cc1OP(=O)(O)O] | 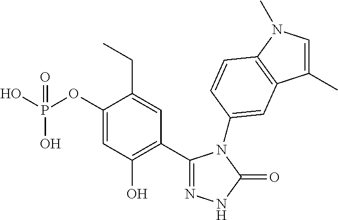 [SMILES: CCc1cc(-c2n[nH]c(=O)n2-c2ccc3c(c2)c(C)cn3C)c(O)cc1OP(=O)(O)O] | 4-(4-(1,3- dimethyl-1H- indol-5-yl)-5- hydroxy-4H- 1,2,4-triazol-3- yl)-2-ethyl-5- hydroxyphenyl dihydrogen phosphate |

Compounds used herein can be prepared according to procedures disclosed in U.S. Publication No. 2006-0167070 and WO2009/023211.

Compounds described herein typically can form a tautomeric structure as shown below and as exemplified by the tautomeric structures shown in Tables 1 and 2:

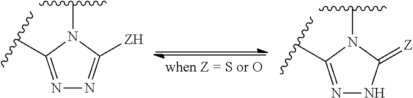

The methods described herein include treating, managing, or ameliorating cancer in a subject in need thereof, where the cancer is mediated through or associated with dysregulated, aberrant, or defective JAK/STAT signaling or one or more symptoms thereof, the methods comprising determining the level of JAK/STAT signaling in a sample derived from a subject with cancer; and administering to the subject an effective amount of a triazolone compound represented by the structural formulae (I) or (Ia) or a compound in Table 1 or Table 2, wherein the presence of dysregulated, aberrant, or defective JAK/STAT signaling is indicated:

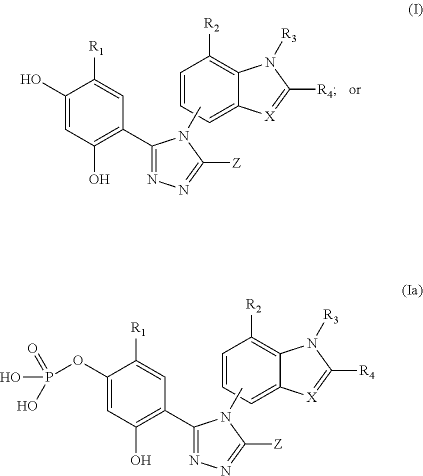

or a tautomer, or a pharmaceutically acceptable salt thereof, wherein:
Z is OH, SH, or NH₂;
X is CR₄ or N;
R₁ is —H, —OH, —SH, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, an alkoxy or cycloalkoxy, a haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂;
R₂ is —H, —OH, —SH, —NR₇H, —OR₁₅, —SR₁₅, —NHR₁₅, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, or —NR₇C(NR₈)NR₁₀R₁₁;
R₃ is —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, a haloalkyl, a heteroalkyl, —C(O)R₇, —(CH₂)ₘC(O)OR₇, —C(O)OR₇, —OC(O)R₇, —C(O)NR₁₀R₁₁, —S(O)ₚR₇, —S(O)ₚOR₇, or —S(O)ₚNR₁₀R₁₁;
R₄ is —H, —OH, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, halo, cyano, nitro, guanadino, a haloalkyl, a heteroalkyl, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, —S(O)ₚNR₁₀R₁₁, or R₄₃ and R₄₄ taken together with the carbon atoms to which they are attached form an optionally substituted cycloalkenyl, an optionally substituted aryl, an optionally substituted heterocyclyl, or an optionally substituted heteroaryl;
R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl;
R₁₅, for each occurrence, is independently, a lower alkyl;
p, for each occurrence, is, independently, 1 or 2; and
m, for each occurrence, is independently, 1, 2, 3, or 4.

In one embodiment, the method also includes improving the efficacy of a triazolone compound in the treatment of a subject with cancer, comprising (a) determining the level of JAK/STAT signaling in a sample derived from the subject; and (b) administering to the subject an effective amount of the triazolone compound represented by the structural formulae (I) or (Ia) as defined above or a compound in Table 1 or Table 2, wherein the presence of dysregulated or aberrant JAK/STAT signaling level is indicated.

In one embodiment, the triazolone compound is 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof. In one embodiment, the triazolone compound is 5-hydroxy-4-(5-hydroxy-4-(1-methyl-1H-indol-5-yl)-4H-1,2,4-triazol-3-yl)-2-isopropylphenyl dihydrogen phosphate dihydrogen phosphate, or a tautomer, or a pharmaceutically acceptable salt thereof. In one embodiment, the triazolone compound is within the range from about 0.15 mg/kg to about 1000 mg/kg. In one embodiment, the triazolone compound is within the range from about 10 mg/kg to about 300 mg/kg. In one embodiment, the triazolone compound is within the range from about 25 mg/kg to about 150 mg/kg. In one embodiment, the triazolone compound is within the range from about 25 mg to about 150 mg. In any one of these embodiments, the cancer may be lung cancer, breast cancer, hematological neoplasms, gastrointestinal stromal tumor, pancreatic cancer, prostate cancer, leukemia, myeloproliferative neoplasms, solid cancer, or non-small cell lung cancer.

In any one of these embodiments, the dysregulated, aberrant, or defective JAK/STAT signaling may be mediated through dysregulated JAK protein. In another embodiment, the dysregulated JAK protein may be mediated through one or more mutations of JAK2 protein.

In any one of these embodiments, the dysregulated, aberrant, or defective JAK/STAT signaling may be mediated through dysregulated STAT3 protein activity. In any one of these embodiments, the dysregulated, aberrant, or defective JAK/STAT signaling may be mediated through dysregulated STAT5 protein activity.

In any one of these embodiments, the dysregulated, aberrant, or defective JAK/STAT signaling may be mediated through dysregulated, aberrant, or defective JAK2/STAT3 signaling.

In any one of these embodiments, the dysregulated, aberrant, or JAK/STAT signaling may be mediated through dysregulated, aberrant, or defective JAK2/STAT5 signaling.

In another embodiment, the method includes administering to the subject with cancer an effective amount of a triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, wherein the presence of dysregulated, aberrant, or defective JAK/STAT signaling in the subject is indicated.

In any one of these embodiments, the cancer may be lung cancer, breast cancer, hematological neoplasms, gastrointestinal stromal tumor, pancreatic cancer, prostate cancer, leukemia, myeloproliferative neoplasms, solid cancer, or non-small cell lung cancer.

In yet another embodiment, the method includes administering to the subject with hematological cancer an effective amount of a triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, wherein the presence of dysregulated, aberrant, or defective JAK2 signaling in the subject is indicated.

In yet another embodiment, the method includes administering to the subject with lung cancer an effective amount of a triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, wherein the presence of dysregulated, aberrant, or defective JAK2/STAT3 signaling in the subject is indicated.

In yet another embodiment, the method includes administering to the subject with non-small cell lung cancer an effective amount of a triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, wherein the presence of dysregulated, aberrant, or defective JAK2/STAT3 signaling in the subject is indicated.

In yet another embodiment, the method includes administering to the subject with acute myeloid leukemia an effective amount of a triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, wherein the presence of dysregulated, aberrant, or defective STAT5 signaling in the subject is indicated.

In any one of the above embodiments, the triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof may be within the range from about 0.15 mg/kg to about 1000 mg/kg. In some of these embodiments, the triazolone compound may be within the range from about 10 mg/kg to about 300 mg/kg. In some of these embodiments, the triazolone compound may be within the range from about 25 mg/kg to about 150 mg/kg. In some of these embodiments, the triazolone compound may be within the range from about 25 mg to about 150 mg.

In yet another embodiment, the method includes administering to the subject with hematological cancer an effective amount of a triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, wherein the presence of dysregulated, aberrant, or defective JAK2 signaling in the subject is indicated. In one embodiment, the triazolone compound is within the range from about 0.15 mg/kg to about 1000 mg/kg. In one embodiment, the triazolone compound is within the range from about 10 mg/kg to about 300 mg/kg. In one embodiment, the triazolone compound is within the range from about 25 mg/kg to about 150 mg/kg. In one embodiment, the triazolone compound is within the range from about 25 mg to about 150 mg.

In yet another embodiment, the method includes administering to the subject with hematological cancer an effective amount of a triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, wherein the presence of dysregulated, aberrant, or defective JAK2 signaling in the subject is indicated.

In yet another embodiment, the method includes administering to the subject with lung cancer an effective amount of a triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, wherein the presence of dysregulated, aberrant, or defective JAK2/STAT3 signaling in the subject is indicated.

In yet another embodiment, the method includes administering to the subject with non-small cell lung cancer an effective amount of a triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, wherein the presence of dysregulated, aberrant, or defective JAK2/STAT3 signaling in the subject is indicated.

In yet another embodiment, the method includes administering to the subject with acute myeloid leukemia an effective amount of a triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, wherein the presence of dysregulated, aberrant, or defective STAT5 signaling in the subject is indicated.

In any one of the above embodiments, the triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a pharmaceutically acceptable salt thereof may be within the range from about 0.15 mg/kg to about 1000 mg/kg. In some of these embodiments, the triazolone compound may be within the range from about 10 mg/kg to about 300 mg/kg. In some of these embodiments, the triazolone compound may be within the range from about 25 mg/kg to about 150 mg/kg. In some of these embodiments, the triazolone compound may be within the range from about 25 mg to about 150 mg.

In one embodiment, the method includes improving the efficacy of a triazolone compound in the treatment of cancer in a subject in need thereof, comprising (a) determining the level of JAK/STAT signaling in a sample derived from the subject; and (b) administering to the subject an effective amount of the triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, wherein the presence of dysregulated or aberrant JAK/STAT signaling level is indicated, and wherein the cancer is selected from the group consisting of lung cancer, breast cancer, hematological neoplasms, gastrointestinal stromal tumor, pancreatic cancer, prostate cancer, leukemia, myeloproliferative neoplasms, solid cancer, and non-small cell lung cancer. In one embodiment, the triazolone compound is within the range from about 0.15 mg/kg to about 1000 mg/kg. In one embodiment, the triazolone compound is within the range from about 10 mg/kg to about 300 mg/kg. In one embodiment, the triazolone compound is within the range from about 25 mg/kg to about 150 mg/kg. In one embodiment, the triazolone compound is within the range from about 25 mg to about 150 mg.

In one embodiment, the method includes improving the efficacy of a triazolone compound in the treatment of cancer in a subject in need thereof, comprising (a) determining the level of JAK/STAT signaling in the subject; and (b) administering to the subject an effective amount of the triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, wherein the presence of dysregulated or aberrant JAK/STAT signaling level in the subject is indicated, and wherein the cancer is selected from the group consisting of lung cancer, breast cancer, hematological neoplasms, gastrointestinal stromal tumor, pancreatic cancer, prostate cancer, leukemia, myeloproliferative neoplasms, solid cancer, and non-small cell lung cancer. In one embodiment, the triazolone compound is within the range from about 0.15 mg/kg to about 1000 mg/kg. In one embodiment, the triazolone compound is within the range from about 10 mg/kg to about 300 mg/kg. In one embodiment, the triazolone compound is within the range from about 25 mg/kg to about 150 mg/kg. In one embodiment, the triazolone compound is within the range from about 25 mg to about 150 mg.

In one embodiment, the method also includes inhibiting or treating cancer or tumor cells in a subject with cancer, comprising (a) determining the level of JAK/STAT signaling in sample from the subject; and (b) administering to the subject an effective amount of a triazolone compound represented by formula (I) or (Ia), or a compound in Table 1 or Table 2, or a tautomer or a pharmaceutically acceptable salt thereof, wherein the presence of dysregulated or aberrant JAK/STAT signaling level is indicated.

In one embodiment, the method the triazolone compound is 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, administered in an amount from about 10 mg/kg to about 300 mg/kg. In one embodiment, the triazolone compound is 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, administered in an amount from about 10 mg/kg to about 300 mg/kg.

In one embodiment, the method the triazolone compound is 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, administered in an amount from about 10 mg to about 300 mg. In one embodiment, the triazolone compound is 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, administered in an amount from about 10 mg to about 300 mg.

In all the aforementioned embodiments, the triazolone compound may be administered intravenously, orally, transdermally, subcutaneously, mucosally, intramuscularly, intranasally, intrapulmonarily, parenterally, intrarectally or topically.

In one embodiment, the method includes inhibiting or treating cancer or tumor cells, comprising (a) determining the level of JAK/STAT signaling in the cancer or tumor cells; and (b) exposing the cells with an effective amount of a triazolone compound represented by formula (I) or (Ia), or a compound in Table 1 or Table 2, or a tautomer or a pharmaceutically acceptable salt thereof, wherein the presence of dysregulated or aberrant JAK/STAT signaling level in the cells is indicated.

In one embodiment, the triazolone compound is 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof. In one embodiment, the triazolone compound is 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof. In one embodiment, the cancer or tumor cells are from the cancer or tumor selected from the group consisting of lung cancer, breast cancer, hematological neoplasms, gastrointestinal stromal tumor, pancreatic cancer, prostate cancer, leukemia, myeloproliferative neoplasms, solid cancer, and non-small cell lung cancer.

The determination of whether or not the level of JAK/STAT signaling in cancer/tumor cells or samples from a subject in need of treatment is normal, dysregulated, aberrant, or defective can be performed by various known biological methods such as western blotting, ELISA, real-time PCR, immunohistochemistry, immunoprecipitation, immunoblotting, multi-analyte profiling beads, flow cytometry according to the procedures published in the art and/or described herein. More particularly, the determination of the level of proteins implicated in the JAK/STAT signaling pathway such as JAK1, JAK2, JAK 3, Tyk2, STAT1, STAT2, STAT3, STAT4, STAT5a, STAT5b, STAT6, and/or their respective phosphorylated forms can be achieved from a sample derived from a subject in need thereof by the aforementioned methods. These methods or techniques are well within the capacity of a person of ordinary skill in the art. For more specific steps and/or procedures, see, e.g., Schoof et al, HSP90 is essential for Jak-STAT signaling in classical Hodgkin lymphoma cells, Cell Communication and Signaling, 7 (1), p. 17, 2009; Holtick et al, STAT3 is essential for Hodgkin lymphoma cell proliferation and is a target of tyrphostin AG17 which confers sensitization for apoptosis, Leukemia 2005, 19:936-944; Marubayashi et al, HSP90 is a therapeutic target in JAK2-dependent myeloproliferative neoplasms in mice and humans, The Journal of clinical investigation, 120 (10), p. 3578-3593, 2010; US application publication 2010/0209929, and all the references cited therein. Even more particularly, the specific information about the determination of whether or not the JAK/STAT signaling pathway is implicated in a patient with breast cancer can be found in the reference by Marotta et al, J Clin Invest. 2011; 121(7):2723-2735, or the references cited therein.

In one embodiment, the method utilizes one or more compounds described herein and at least one other therapy which has the same mechanism of action as the compounds. In another embodiment, the method utilizes one or more compounds described herein and at least one other therapy which has a different mechanism of action than the compounds. In certain embodiments, the methods improve the therapeutic effect of one or more compounds described herein by functioning together with other therapies to have an additive or synergistic effect. In certain embodiments, the methods reduce the side effects associated with the therapies. In certain embodiments, the methods reduce the effective dosage of one or more of the therapies.

In one embodiment, the method utilizes a pharmaceutical composition comprising one or more compounds described herein to treat a subject, preferably a human, to prevent, treat, manage, or ameliorate cancer, or one or more symptom thereof. In another embodiment, the pharmaceutical composition described herein may also comprise one or more other agents being used, have been used, or are known to be useful in the treatment or amelioration of cancer or a symptom thereof.

The methods also include managing, treating or ameliorating cancer, or one or more symptoms thereof in a subject refractory, either completely or partially, to existing agent therapies for cancer, the methods comprising determining the level of JAK/STAT signaling in a sample from the subject; and administering to the subject an effective amount of a triazolone compound represented by the structural formulae (I) or (Ia) or a compound in Table 1 or Table 2, and a dose of an effective amount of one or more therapies, wherein the presence of dysregulated, aberrant, or defective JAK/STAT signaling in indicated. The methods also include treating, managing, or ameliorating cancer, or a symptom thereof, where the cancer is mediated through dysregulated, aberrant, or defective JAK/STAT signaling, by administering one or more compounds described herein in combination with any other therapy(ies) to patients who have proven refractory to other therapies but are no longer on these therapies.

The compounds described herein and/or other therapies can be administered to a subject by any route known to one of skill in the art. Examples of routes of administration include, but are not limited to, parenteral, e.g., intravenous, intradermal, subcutaneous, oral (e.g., inhalation), intranasal, transdermal (topical), transmucosal, and rectal administration.

The methods utilize pharmaceutical compositions for the treatment, and amelioration of JAK/STAT mediated cancer. In one embodiment, the composition comprises one or more compounds described herein, or a pharmaceutically acceptable salt thereof. In another embodiment, the composition described herein comprises one or more therapeutic agents other than a compound described herein, or a pharmaceutically acceptable salt. In another embodiment, the composition described herein comprises one or more compounds described herein, or a pharmaceutically acceptable salt thereof, and one or more other therapeutic agents. In another embodiment, the composition comprises a compound described herein, or a pharmaceutically acceptable salt, thereof, and a pharmaceutically acceptable carrier, diluent or excipient. Suitable carriers, diluents, or excipients are well known to those skilled in the art of pharmacy.

A pharmaceutical composition described herein is formulated to be compatible with its intended route of administration. Examples of routes of administration include, but are not limited to, parenteral, e.g., intravenous, intradermal, subcutaneous, oral (e.g., inhalation), intranasal, transdermal (topical), transmucosal, and rectal administration. In a specific embodiment, the composition is formulated in accordance with routine procedures as a pharmaceutical composition adapted for intravenous, subcutaneous, intramuscular, oral, intranasal or topical administration to human beings. In a preferred embodiment, a pharmaceutical composition is formulated in accordance with routine procedures for subcutaneous administration to human beings.

The triazolone compounds described herein can be also formulated into or administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566.

In general, the recommended daily dose range of a compound described herein for the conditions described herein lie within the range of from about 0.01 mg to about 1000 mg per day, given as a single once-a-day dose preferably as divided doses throughout a day. In one embodiment, the daily dose is administered twice daily in equally divided doses. Specifically, a daily dose range should be from about 5 mg to about 500 mg per day, more specifically, between about 10 mg and about 200 mg per day. In managing the patient, the therapy should be initiated at a lower dose, perhaps about 1 mg to about 25 mg, and increased if necessary up to about 200 mg to about 1000 mg per day as either a single dose or divided doses, depending on the patient's global response. It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with individual patient response.

Different therapeutically effective amounts may be applicable, as will be readily known by those of ordinary skill in the art. Similarly, amounts sufficient to prevent, manage, treat or ameliorate such proliferative disorders, but insufficient to cause, or sufficient to reduce, adverse effects associated with the compounds described herein are also encompassed by the above described dosage amounts and dose frequency schedules. Further, when a patient is administered multiple dosages of a compound described herein, not all of the dosages need be the same. For example, the dosage administered to the patient may be increased to improve the prophylactic or therapeutic effect of the compound or it may be decreased to reduce one or more side effects that a particular patient is experiencing.

In one embodiment, the dosage of the composition or a compound described herein administered to prevent, treat, manage, or ameliorate a cancer, or one or more symptoms thereof in a patient is 150.1 g/kg, preferably 250 μg/kg, 500 μg/kg, 1 mg/kg, 5 mg/kg, 10 mg/kg, 25 mg/kg, 50 mg/kg, 75 mg/kg, 100 mg/kg, 125 mg/kg, 150 mg/kg, or 200 mg/kg or more of a patient's body weight. In another embodiment, the dosage of the composition or a compound described herein administered to prevent, treat, manage, or ameliorate cancer, or one or more symptoms thereof in a patient is a unit dose of 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7 mg, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg. The unit dose can be administered 1, 2, 3, 4 or more times daily, or once every 2, 3, 4, 5, 6 or 7 days, or once weekly, once every two weeks, once every three weeks or once monthly.

The dosages of prophylactic or therapeutic agents other than compounds described herein, which have been or are currently being used to prevent, treat, manage, or proliferative disorders, such as cancer, or one or more symptoms thereof can be used in the combination with the method describer herein. Preferably, dosages lower than those which have been or are currently being used to prevent, treat, manage, or ameliorate a proliferative disorder, or one or more symptoms thereof, are used in the combination. The recommended dosages of agents currently used for the prevention, treatment, management, or amelioration of a proliferative disorders, such as cancer, or one or more symptoms thereof, can obtained from any reference in the art including Hardman et al., eds., 1996, Goodman & Gilman's The Pharmacological Basis Of Basis Of Therapeutics 9^{th} Ed, Mc-Graw-Hill, New York; Physician's Desk Reference (PDR) 57^{th} Ed., 2003, Medical Economics Co., Inc., Montvale, N.J.

In certain embodiments, when the compounds described herein are administered in combination with another therapy, the therapies (e.g., prophylactic or therapeutic agents) are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In one embodiment, two or more therapies (e.g., prophylactic or therapeutic agents) are administered within the same patent visit.

In certain embodiments, one or more compounds described herein and one or more other the therapies (e.g., therapeutic agents) are cyclically administered. Cycling therapy involves the administration of a first therapy (e.g., a first prophylactic or therapeutic agents) for a period of time, followed by the administration of a second therapy (e.g., a second prophylactic or therapeutic agents) for a period of time, followed by the administration of a third therapy (e.g., a third prophylactic or therapeutic agents) for a period of time and so forth, and repeating this sequential administration, i.e., the cycle in order to reduce the development of resistance to one of the agents, to avoid or reduce the side effects of one of the agents, and/or to improve the efficacy of the treatment.

In certain embodiments, administration of the same compound described herein may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated and the administration may be separated by at least at least 1 day, 2 days, 3 days, 5 days, 10 days, 15 days, 30 days, 45 days, 2 months, 75 days, 3 months, or 6 months.

In another embodiment, the method includes preventing, treating, managing, or ameliorating a proliferative disorders, such as cancer, or one or more symptoms thereof, the methods comprising administering to a subject in need thereof a dose of at least 150 μg/kg, preferably at least 250 μg/kg, at least 500 μg/kg, at least 1 mg/kg, at least 5 mg/kg, at least 10 mg/kg, at least 25 mg/kg, at least 50 mg/kg, at least 75 mg/kg, at least 100 mg/kg, at least 125 mg/kg, at least 150 mg/kg, or at least 200 mg/kg or more of one or more compounds described herein once every day, preferably, once every 2 days, once every 3 days, once every 4 days, once every 5 days, once every 6 days, once every 7 days, once every 8 days, once every 10 days, once every two weeks, once every three weeks, or once a month. Alternatively, the dose can be divided into portions (typically equal portions) administered two, three, four or more times a day.

Embodiments of the invention, showing the efficacy of these triazolone compounds type of compounds in treating JAK/STAT mediated cancers, are illustrated by the following non-limiting examples.

### Experimental Protocols

The synthetic small molecule Hsp90 inhibitor ganetespib was employed as an exemplary compound. Ganetespib binds in the ATP-binding domain at the N-terminus of Hsp90. In preclinical studies, the drug showed low nanomolar activity in vitro against a variety of human cancer cell lines and potent antitumor efficacy against human xenografts models. It was found that ganetespib potently induced apoptosis in a variety of tumor lines dependent on persistent JAK/STAT signaling for growth and survival. It was further demonstrated that the drug also altered many elements of cell cycle regulation in the cancer cells, an activity absent from a JAK-specific inhibitor. In vivo, ganetespib's coordinate impact on both cell growth and cell division resulted in potent antitumor activity in JAK/STAT-driven models of human leukemia. As such, these triazolone compounds may effectively be used to treat diseases dependent on constitutive JAK/STAT signaling, such as lung cancer, breast cancer, hematological neoplasms, gastrointestinal stromal tumor, pancreatic cancer, prostate cancer, leukemia, myeloproliferative neoplasms, solid cancer, or non-small cell lung cancer.

### Cell Culture

All cell lines were obtained from the ATCC (Rockville, Md., USA), with the exception of SET-2 cells which were purchased from the German Collection of Microorganisms and Cell Cultures (DSMZ, Germany). Cells were maintained and cultured according to standard techniques at 37° C. in 5% (v/v) CO₂ using culture medium recommended by the supplier.

### Reagents

Hsp90 inhibitors ganetespib and 17-AAG (synthesized at Synta Pharmaceuticals Corp.), the JAK inhibitor Pyridone 6 (Calbiochem, Darmstadt, Germany), and proteasome inhibitors MG-132 and lactacystin (Boston Biochem, Cambridge, Mass., USA) were dissolved in dimethyl sulfoxide (DMSO), aliquotted and stored at −20° C. All primary antibodies were purchased from Cell Signaling Technology (CST, Beverly, Mass., USA) with the exception of JAK1 (Santa Cruz Biotechnology, Santa Cruz, Calif., USA) and STATS (Epitomics, Burlingame, Calif., USA). Secondary antibodies were purchased from LI-COR (Lincoln, Nebr., USA).

### Cell Viability Assays

Cells were grown in 96-well plates based on optimal growth rates determined empirically for each line. Twenty-four hours after plating, cells were dosed with the indicated compound or DMSO (0.3%) for 72 h. AlamarBlue (Invitrogen, Carlsbad, Calif., USA) was added (10% v/v) to the cells, and the plates were incubated for 3 h and subjected to fluorescence detection (Ex=545 nm, Em=600 nm) in a SpectraMax Plus 384 microplate reader (Molecular Devices, Sunnyvale, Calif., USA). Data are normalized to percent of control, with IC50 values based on a four-parameter logistic dose response curve (model 205) using XLfit version 5.1 from IDBS (Guildford, U.K.).

### Western Blotting

Cells were disrupted in lysis buffer (CST) and solubilized in 2% SDS. Xenograft tumors (average volume of 100-200 mm3) were excised, cut in half, and flash frozen in liquid nitrogen. Each tumor fragment was lysed in 0.5 mL of lysis buffer using a FastPrep-24 homogenizer and Lysing Matrix A (MP Biomedicals, Solon, Ohio, USA) and lysates clarified by centrifugation. Equal amounts of proteins were resolved by SDS-PAGE and immunoblotted with indicated antibodies. The antigen-antibody complex was visualized and quantitated using an Odyssey system (LI-COR, Lincoln, Nebr., USA).

### Real Time Quantitative RT-PCR

HEL92.1.7 cells were treated with DMSO, P6 (100 or 1000 nM) or ganetespib (25 or 250 nM) for 4 h or 24 h, and total RNA extracted using Trizol (Invitrogen). RNA was purified with the RNeasy kit (Qiagen, Hilden, Germany) and converted to cDNA via an iScript kit (BioRad, Hercules, Calif., USA). Oligonucleotide primers were purchased from SABiosciences (Frederick, Md., USA). PCR was done with iQ SYBR Green Supermix (Bio-Rad), and levels of the tested genes were normalized to HPRT expression using the comparative Ct method.

### Affymetrix Gene Expression Analysis

Biotinylated aRNA was generated by in vitro transcription using Affymetrix GeneChip Expression IVT labeling kit (Affymetrix, Santa Clara, Calif., USA). Fifteen micrograms of labeled aRNA was fragmented and hybridized to Affymetrix GeneChip Human Genome U133 Plus 2 arrays and scanned using a GeneChip Scanner (Affymetrix). Array data were analyzed with the Affymetrix Expression Console Software utilizing the MASS algorithm. In order to generate a threshold for identifying probe sets that have large differences between 1000 nM P6 (4 h) and 250 nM ganetespib (24 h) arrays and their controls in the absence of treatment replicates, data from four control arrays (DMSO only; two at 4 h, and two at 24 h) were used to create an expression level dependent fold-difference envelope that reflects the increasing measurement variability as expression level decreases. This envelope, used in lieu of a fixed fold-difference criteria, was formed by identifying the xth percentile expression-level difference (where x was large, typically 99.9%) at each mean expression level bin. To achieve this, data were used from the six possible comparisons from the four DMSO-only arrays. The resulting, smoothed threshold fold-difference envelope was then applied to the two compound-treated/control array pairs to identify those probe-sets that have large expression level differences between treatment and control. For hierarchical analysis, genes with greater than two fold changes in expression with 250 nM ganetespib were clustered using established algorithms in Cluster (49) and a subset visualized by TreeView.

### Flow Cytometry

HEL92.1.7 cells were plated at 0.5×106 cells/mL and treated as indicated. Cells were harvested and stained with propidium iodide using the BD Cycle TEST PLUS Reagent Kit (BD Biosciences, San Jose, Calif., USA) according to the manufacturer's instructions. Twenty thousand cells were analyzed for their DNA content using a FACS Caliber cytometer (BD Biosciences).

### In Vivo Leukemia Xenograft Models

Eight-week-old female immunodeficient CB-17/Icr-Prkdcscid/Crl (SCID) mice (Charles River Laboratories, Wilmington, Mass.) were maintained in a pathogen-free environment, and all in vivo procedures were approved by the Synta Pharmaceuticals Corp. Institutional Animal Care and Use Committee. For the MV4-11 model, tumor cells were subcutaneously implanted in SCID mice as previously described (34). Tumor volumes (V) were calculated by caliper measurements of the width (W), length (L), and thickness (T) of each tumor using the formula: V=0.5236 (LWT).

Animals with 100-200 mm³ tumors were then randomized into treatment groups of 8 and i.v. dosed via the tail vein at 10 mL/kg body weight with either vehicle or ganetespib formulated in 10/18 DRD (10% DMSO, 18% Cremophor RH 40, 3.6% dextrose, 68.4% water). Tumor growth inhibition was monitored by tumor volume measurements twice weekly. As a measurement of in vivo efficacy, the % T/C value was determined from the change in average tumor volumes of each treated group relative to the vehicle-treated or itself in the case of tumor regression. Statistical significance was determined using a Kruskal-Wallis one-way ANOVA followed by the Tukey Test multiple comparison procedure.

For the HEL92.1.7 model, SCID mice were i.v. injected via the tail vein with 5×106 cells in phosphate-buffered saline (PBS) on day 0. Implanted animals were then randomized into groups of 10 and i.v. dosed via the tail vein at 10 mL/kg body weight with either vehicle or ganetespib formulated in 10/18 DRD. Animals were weighed daily and removed from the study at the first sign of hind limb paralysis, which occurred in 100% of vehicle-treated animals. Median overall survival was estimated using the Kaplan-Meier method and the log-rank test (2-sided) for statistical significance. Tumor cells were identified as either CD44- or CD54-positive cells within the CD45-negative population. Spinal column tumor burden was determined by quantitative image analysis with ImageJ software (Wayne Rasband, National Institute of Mental Health) of hematoxylin and eosin stained tissue sections.

For both models, studies were conducted at the highest non-severely toxic doses (NHSTDs) of 150 mg/kg ganetespib one-time per week or 25 mg/kg ganetespib 5×/week for 3 weeks based on prior tolerability studies conducted in non-tumor bearing mice.

### Example 1

### Ganetespib Inhibition of JAK2-Mediated Signal Transduction and Proliferation in Hematological Cancers

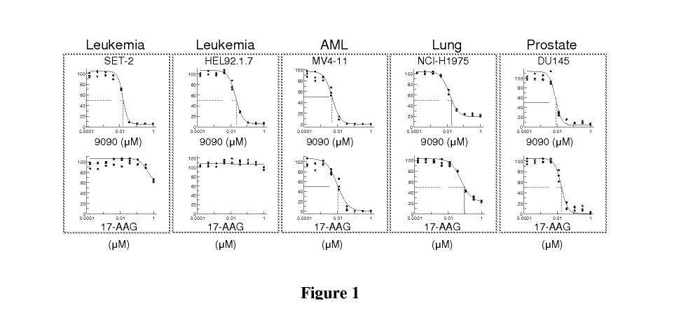

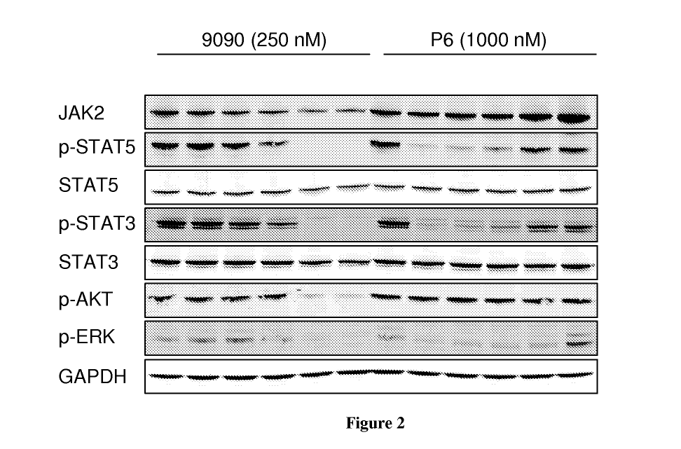

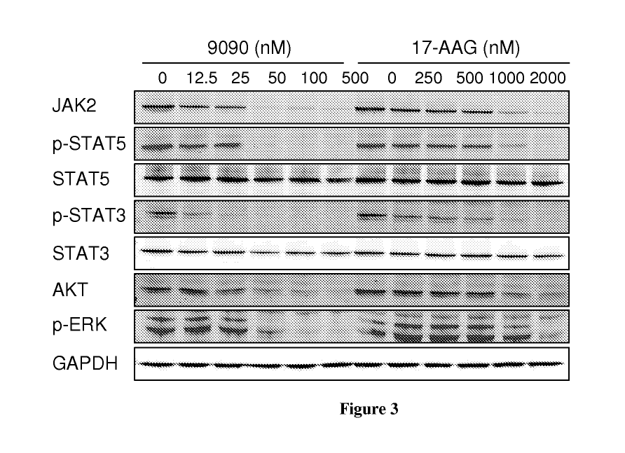

Ganetespib has low nanomolar potency and reduced cellular viability in a group of human hematological and solid tumor cell lines selected for their dependence on JAK/STAT signaling and varying cancer type (FIG. 1). In each of the lines tested, ganetespib was more potent than the ansamycin Hsp90 inhibitor 17-AAG. Ganetespib was greater than 100 fold more potent than 17-AAG in the SET-2 and HEL92.1.7 leukemia cells, cell lines harboring constitutively active JAK2V617F mutations that act as their oncogenic drivers. Using the HEL92.1.7 cells, the comparison was made of the JAK/STAT inhibitory activity of ganetespib with the compound Pyridone-6 (P6), a reversible, ATP-competitive pan inhibitor of the JAKs (FIG. 2). It can be seen that ganetespib and P6 each blocked JAK2 dependent signaling, as evidenced by the loss of phospho-STAT3 and phospho-STAT5, and ERK signaling. However, ganetespib was at least four-fold more potent and suppressed STAT signaling longer when compared to P6. Also distinguishing the two compounds was that ganetespib treatment alone led to the targeted loss of JAK2 and phospho-AKT protein levels (FIG. 2), both Hsp90 client proteins. Ganetespib treatment resulted in sustained inhibition of multiple oncogenic targets in these cellular models of JAK2-driven malignancy. Similar effects on JAK/STAT signaling were seen with SET-2 cells, where 50 nM ganetespib was able to destabilize JAK2 sufficiently to result in loss of activated (i.e., phosphorylated) STAT3 and STAT5 expression (FIG. 3). 17-AAG showed comparable effects as ganetespib, but was 200 fold less potent, in line with the viability data described above. Taken together, these data demonstrated that ganetespib has superior JAK/STAT inhibitory activity to both P6 and 17-AAG in terms of potency and duration of response.

### Example 2

### Ganetespib Abrogates JAK/STAT Signaling in Solid Tumors

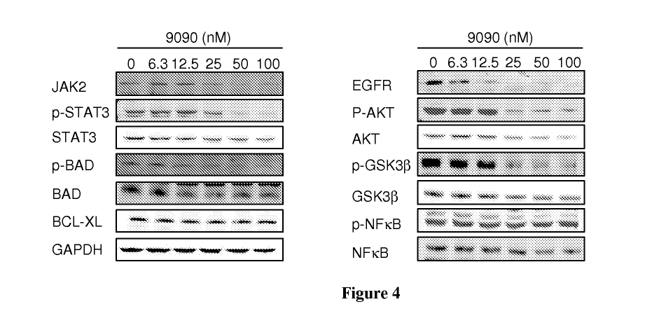

In addition to its incidence in hematologic malignancies, oncogenic STAT activation is also prevalent in a range of solid tumors. For example, persistently activated STAT3 is found in 50% of lung adenocarcinomas and is primarily observed in tumors harboring somatic-activating mutations in the epidermal growth factor receptor (EGFR). The NCI-H1975 non-small cell lung cancer (NSCLC) cell line expresses the Hsp90 client EGFRL858R/T790M, a constitutively activated and erlotinib-resistant form of EGFR, and ganetespib treatment resulted in a dose dependent decrease in EGFR expression in these cells (FIG. 4). Moreover, ganetespib also induced potent degradation of JAK2 and loss of phosphorylated STAT3 in a dose-dependent manner. Inactivation of AKT and GSK3β, proteins important in regulating apoptosis, was observed with a similar dose response to that of JAK2/STAT3 signaling. It has been shown that JAK2 can modulate the activity of additional apoptotic regulators such as BAD and BCL-XL to promote cell survival. Consistent with this, a concomitant reduction in the levels of phosphorylated BAD (FIG. 4) was found, thus reducing the pro-apoptotic activity of this protein. These data suggested a potential mechanism to account for the cytotoxic response observed with ganetespib treatment (FIG. 1).

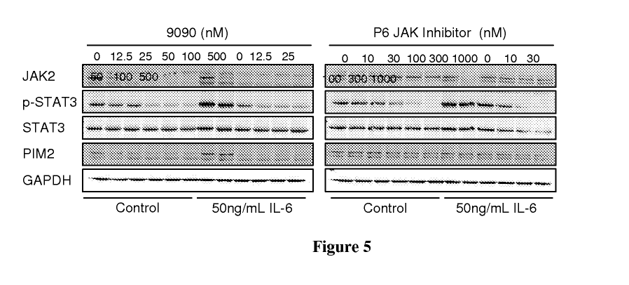

The JAK/STAT signaling axis is a key modulator of cytokine signaling in both normal and transformed cells and one proposed mechanism for aberrant STAT3 activation in lung cancer involves the up-regulation of autocrine and/or paracrine IL-6 signaling. It was found that in the absence of external ligand, HCC827 cells treated with ganetespib exhibited a dose-dependent decrease in JAK2 expression, leading to a loss of STAT3 activity and expression of the downstream STAT target PIM2 (FIG. 5). Biochemical inhibition of JAK2 by P6, albeit at higher concentrations, similarly downregulated constitutive STAT3 activity but did not influence total JAK2 protein levels. Similarly, both compounds blocked JAK/STAT signaling stimulation when the pathway was activated by exogenous IL-6 treatments (FIG. 5).

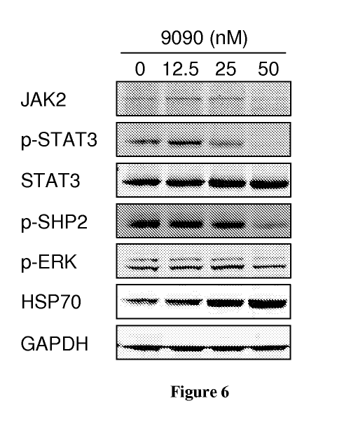

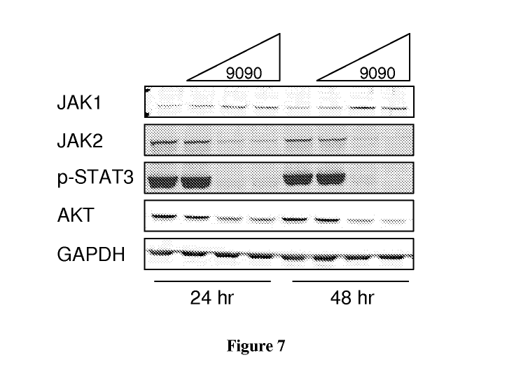

Dysregulated IL-6/JAK2 signaling has also been implicated in prostate cancer tumorigenesis. In this regard, the DU145 prostate cancer cell line expresses an autocrine IL-6 signaling loop and has been reported to be sensitive to the effects of a novel small molecule JAK2 inhibitor in vitro and in vivo. Ganetespib was a potent inducer of cell death in this line (FIG. 1). Biochemical characterization of DU145 cells revealed similar inhibitory effects on JAK2 signaling following ganetespib treatment (FIG. 6). Loss of JAK2, phospho-STAT3 and phospho-SHP2, a JAK2 interacting phosphatase important for JAK2 signal transduction, was observed following addition of ganetespib. The related JAK1 kinase expressed in this cell line was not targeted for degradation but instead appeared to increase following ganetespib exposure (FIG. 7). Analogous results were obtained for the PC-3 prostate cancer cell line. These data showed that selective degradation of JAK2 in DU145 prostate cells was sufficient to abrogate subsequent activation of STAT3 signaling.

### Example 3

### Hsp90 Inhibition Downregulated Transcription of JAK/STAT Signaling Targets and Cell Cycle Genes

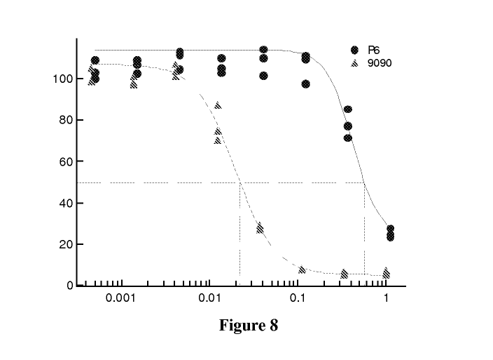

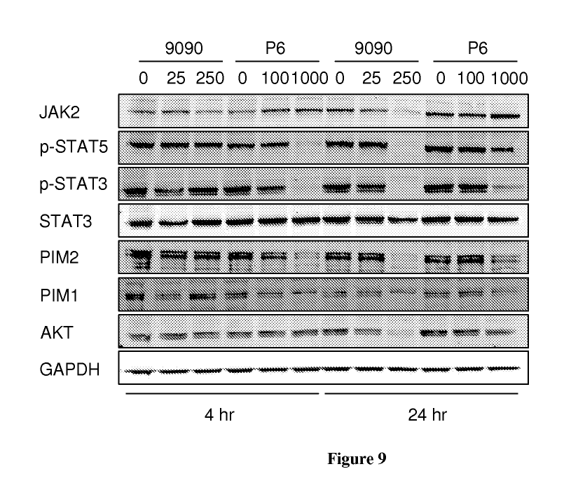

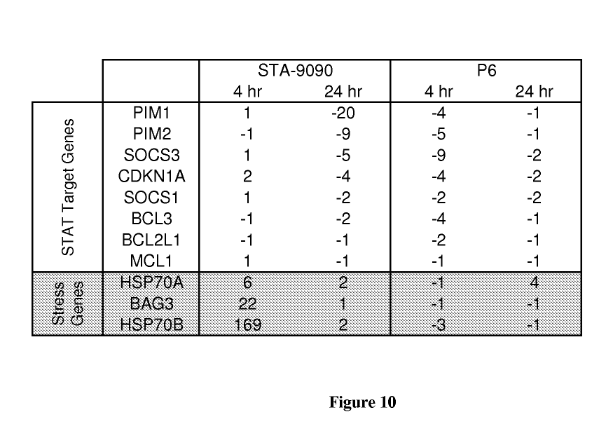

In HEL92.1.7 erythroleukemia cells, biochemical inhibition of JAK2 by P6 treatment resulted in a loss of cellular viability, but with 30 fold less potency than ganetespib (IC₅₀ values 614 vs. 20 nM) (FIG. 8). To compare the cellular impact of each inhibitor, conditions were identified under which JAK2 activity was reduced to equivalent levels by each drug based on their kinetic and potency differences. As illustrated in FIG. 9, the 4 hour P6 (1000 nM) and 24 hour ganetespib (250 nM) treatments were selected because of comparable effects on STAT3/5 signaling. RNA expression profiling at these time points revealed that many JAK/STAT target genes, such as SOCS and PIM family members, were downregulated by both drugs. However, additional genes were altered by ganetespib treatment that was unaffected in the P6-treated cells. Besides leading to the up-regulation of numerous heat shock protein genes, ganetespib treatment also selectively altered the expression of a large set of genes involved in cell cycle-related activities, including DNA replication and repair (BRCA1/2), cell cycle regulation (CDC2, CDC25), centrosome/spindle activities (BUB1/3, CENPE/M, KIF14, FAM33A), chromosome condensation (TOP2A, NCAPG), and replication (RFC3/4, MCM family). Indeed, analysis of the altered genes by hierarchical clustering and enrichment score revealed that modulators of cell division were the most prominent processes diminished by ganetespib treatment (FIG. 10).

### Example 4

### Modulation of Cell Cycle Protein Expression by Ganetespib Induces Growth Arrest

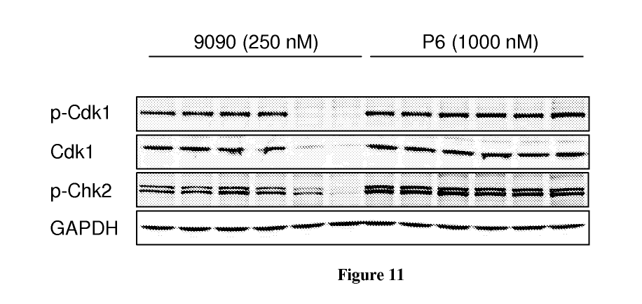

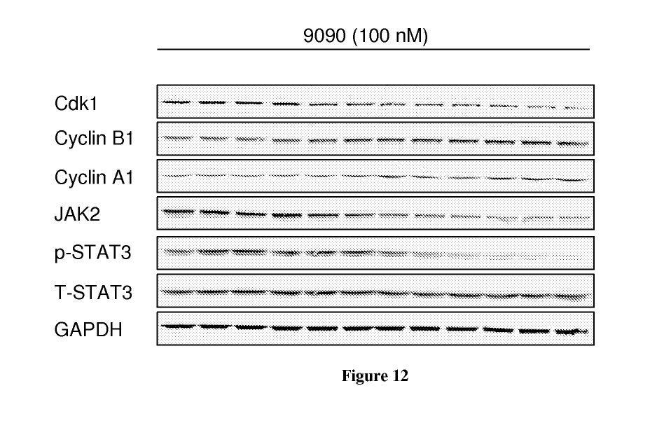

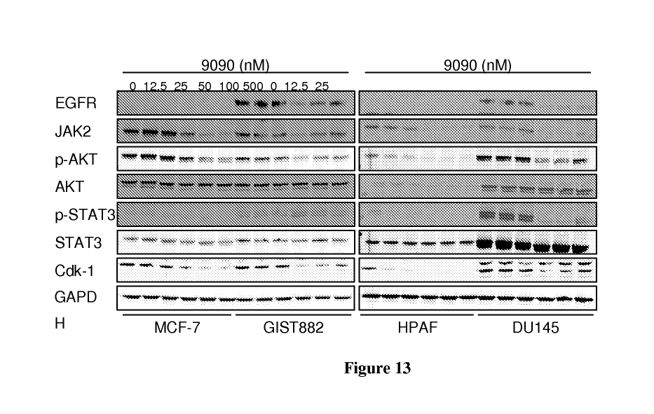

It was also found that ganetespib induced a temporal G1 and G2/M arrest in HEL92.1.7 cells, with concomitant loss of S phase. In contrast, P6 treatment induced accumulation in G1 phase only, without the loss of S phase or G2/M arrest. The targeted effects of ganetespib were examined on critical mediators of cell cycle division at the protein level. Reduced protein levels of cyclin dependent kinase 1 (Cdk-1), a key regulator of the G2/M checkpoint was observed, following a 24 hour exposure to ganetespib, an effect that persisted until at least 48 hours (FIG. 11). In contrast, P6 had no effect on Cdk1 expression. Further, the level of phospho-Chk2, another integral checkpoint kinase, was reduced by ganetespib treatment. As shown in FIG. 12, the destabilization of cyclin kinases was also associated with a temporal accumulation of cyclins A1 and B1 in response to drug addition. Moreover, these effects of ganetespib on both JAK2/STAT signaling and cell cycle regulation were observed in additional cancer types, including breast (MCF-7), gastrointestinal stromal (GIST882), pancreatic (HPAF) and prostate (DU145) tumor cell lines (FIG. 13). Overall, these additional influences on the cell division machinery suggest that ganetespib possessed decided advantages over JAK-specific inhibitors for controlling STAT-driven malignancies.

### Example 5

### Ganetespib Prolongs Survival in a JAK2V617F-Mutant Mouse Model of Human Leukemia

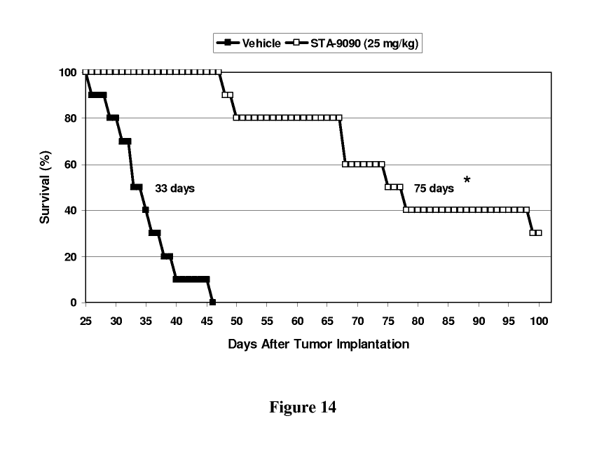

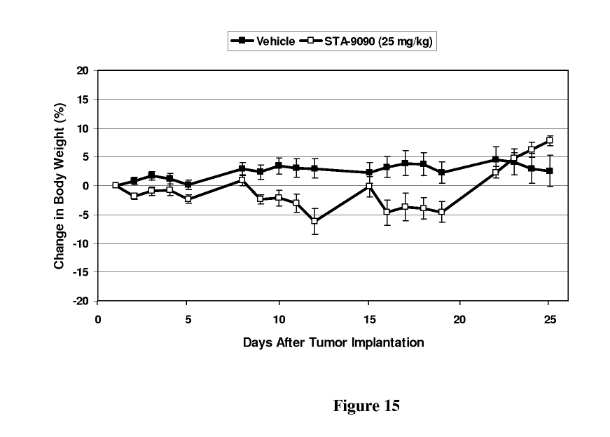

To determine whether these dual activities of ganetespib on JAK2/STAT signaling and cell cycle progression observed in vitro translate into antitumor efficacy in vivo, an orthotopic leukemia model using HEL92.1.7 cells was established. This resulted in the development of disseminated disease with morbidity typically resulting from hind limb paralysis caused by spinal column metastases. To study the effect of ganetespib on survival, beginning one day after tumor cell implantation, the drug was dosed intravenously at its highest non-severely toxic dose (HNSTD) of 25 mg/kg on a 5×/week schedule through day 19. As shown in FIG. 14, ganetespib treatment more than doubled median overall survival (76.5 days vs. 34 days, P<0.0001). The ganetespib treatment was well tolerated, with no significant loss of body weight found after 3 weeks of dosing (FIG. 15). The increased survival of the treated animals correlated with dramatically decreased tumor cell burden in their bone marrow and spinal cord, as determined by histological analysis.

### Example 6

### Ganetespib Exhibits Potent In Vivo Efficacy in STAT5 Driven AML Xenografts

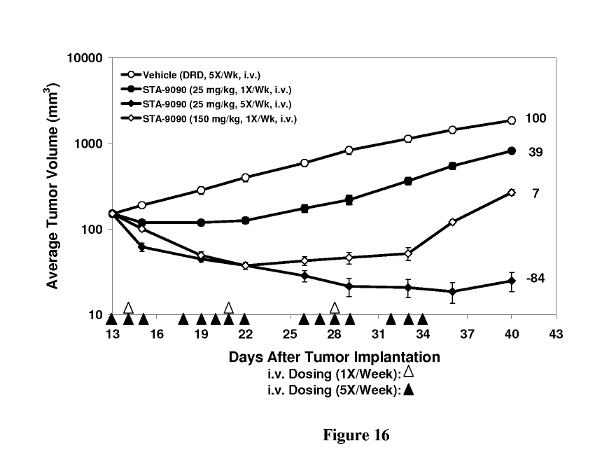

MV4-11 acute myeloid leukemia cells express constitutive STATS activity as a consequence of an internal tandem duplication (ITD) mutation in the FLT3 receptor tyrosine kinase, another Hsp90 client protein and, as such, represent an alternative model of STAT-driven oncogenesis. These cells were highly sensitive to ganetespib in vitro (FIG. 1) and their dose response to ganetespib treatments in xenografts was evaluated. Ganetespib was intravenously administered to tumor-bearing SCID mice at either the daily or weekly HNSTD of 25 mg/kg or 150 mg/kg, respectively. As shown in FIG. 16, the weekly treatment schedule resulted in significant and dose dependent tumor growth inhibition, while the daily dosing regimen (25 mg/kg 5×/week, as used in the orthotopic model above) resulted in significant tumor regression (84%). In both dosing regimens, tumor growth was suppressed for up to a week or more once treatment was discontinued. Beyond this period, as evidenced by the once-per-week treatment cohort, tumor growth could re-initiate.

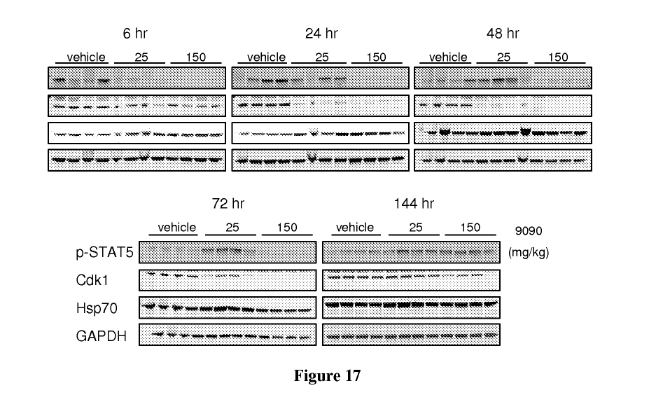

To determine whether these tumor responses correlated with target modulation in vivo, additional mice bearing MV4-11 xenografts were treated with a single dose of vehicle alone or ganetespib at 25 or 150 mg/kg. Tumors were harvested between 6 and 144 hours later and pharmacodynamic analysis was performed by examining the expression levels of phospho-STAT5, Cdk1 and Hsp70 (FIG. 17). In accord with the in vivo tumor growth data, dose-dependent effects on the duration of target inhibition within the tumors were observed. A single 150 mg/kg dose of ganetespib repressed activation of STATS and suppressed expression of Cdk1 for more than three days, consistent with its efficacy in once-per-week dosing. At 25 mg/kg, potent inhibition of STATS activity was achieved within 6 hours as was loss of Cdk1 at 24 hours following ganetespib administration. It was found that STAT5 activity recovered in these tumors by 24 hours, while Cdk1 expression remained suppressed through at least 48 hours even with this low dose (FIG. 17). While the relatively quick recovery of STAT5 activation should have allowed the tumor to restart growth, the more durable suppression of the cell cycle regulators appeared to have kept the growth of the tumors arrested until the next drug dosing.

All publications, patent applications, patents, and other documents cited herein are incorporated by reference in their entirety. In case of conflict, the present specification, including definitions, will control. In addition, the materials, methods, and examples throughout the specification are illustrative only and not intended to be limiting in any way. While the invention has been described and illustrated with reference to particular embodiments thereof, those skilled in the art will appreciate that various adaptations, changes, modifications, substitutions, deletions, or additions of procedures and protocols may be made without departing from the spirit and scope of the invention. It is intended, therefore, that the invention be defined by the scope of the claims that follow and that such claims be interpreted as broadly as is reasonable.

What is claimed is:

1-26. (canceled)

27. A method of improving the efficacy of a triazolone compound in the treatment of cancer in a subject in need thereof, comprising:
a) determining the level of JAK/STAT signaling in a sample derived from the subject; and
b) administering to the subject an effective amount of the triazolone compound of 3-(2,4-dihydroxy-5-isopropyl-phenyl)-4-(1-methyl-indol-5-yl)-5-hydroxy-[1,2,4]triazole, or 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, wherein the presence of dysregulated or aberrant JAK/STAT signaling level is indicated.

28. The method of claim 27, wherein the cancer is selected from the group consisting of lung cancer, breast cancer, hematological neoplasms, gastrointestinal stromal tumor, pancreatic cancer, prostate cancer, leukemia, myeloproliferative neoplasms, solid cancer, and non-small cell lung cancer.

29. The method of claim 27, wherein the dysregulated or aberrant JAK/STAT signaling is mediated through dysregulated JAK protein.

30. The method of claim 29, wherein the dysregulated JAK protein is mediated through mutation of JAK2 protein.

31. The method of claim 27, wherein the dysregulated or aberrant JAK/STAT signaling is mediated through dysregulated STAT3 protein activity.

32. The method of claim 27, wherein the dysregulated or aberrant JAK/STAT signaling is mediated through dysregulated STAT5 protein activity.

33. The method of claim 27, wherein the dysregulated or aberrant JAK/STAT signaling is mediated through dysregulated or aberrant JAK2/STAT3 signaling.

34. The method of claim 27, wherein the dysregulated or aberrant JAK/STAT signaling is mediated through dysregulated or aberrant JAK2/STAT5 signaling.

35. The method of claim 27, wherein the effective amount of the triazolone compound administered is within the range from about 0.15 mg/kg to about 1000 mg/kg.

36. The method of claim 35, wherein the effective amount of the triazolone compound administered is within the range from about 10 mg/kg to about 300 mg/kg.

37. The method of claim 36, wherein the effective amount of the triazolone compound administered is within the range from about 25 mg/kg to about 150 mg/kg.

38. A method of inhibiting or treating cancer or tumor cells in a subject with cancer, comprising:
a) determining the level of JAK/STAT signaling in sample from the subject; and
b) administering to the subject an effective amount of the triazolone compound of 3-(2,4-dihydroxy-5-isopropyl-phenyl)-4-(1-methyl-indol-5-yl)-5-hydroxy-[1,2,4]triazole, or 5-hydroxy-4-(5-hydroxy-4-(1-methyl-1H-indol-5-yl)-4H-1,2,4-triazol-3-yl)-2-isopropylphenyl dihydrogen phosphate, or a tautomer or a pharmaceutically acceptable salt thereof, wherein the presence of dysregulated or aberrant JAK/STAT signaling level is indicated.

39. The method of claim 38, where the cancer is selected from the group consisting of lung cancer, breast cancer, hematological neoplasms, gastrointestinal stromal tumor, pancreatic cancer, prostate cancer, leukemia, myeloproliferative neoplasms, solid cancer, and non-small cell lung cancer.

40. The method of claim 38, wherein the triazolone compound or a tautomer, or a pharmaceutically acceptable salt thereof, is administered in an amount from about 10 mg/kg to about 300 mg/kg.

41. A method of inhibiting or treating cancer or tumor cells, comprising:
a) determining the level of JAK/STAT signaling in the cancer or tumor cells; and
b) exposing the cancer or tumor cells with an effective amount of a triazolone compound of 3-(2,4-dihydroxy-5-isopropyl-phenyl)-4-(1-methyl-indol-5-yl)-5-hydroxy-[1,2,4]triazole, or 5-hydroxy-4-(5-hydroxy-4-(1-methyl-H-indol-5-yl)-4H-1,2,4-triazol-3-yl)-2-isopropylphenyl dihydrogen phosphate, or a tautomer or a pharmaceutically acceptable salt thereof, wherein the presence of dysregulated or aberrant JAK/STAT signaling level in the cells is indicated.
